(19) United States
(12) Patent Application Publication
(10) Pub. No.: US 2008/0243053 A1
(43) Pub. Date: Oct. 2, 2008

(54) METHOD FOR INSERTING A CANNULA INTO AN INFUSION SITE

(75) Inventors: Mark A. DeSTEFANO, Collegeville, PA (US); Jeff Kiersh, Exton, PA (US); Steven E. Wojcik, Shoreline, WA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Animas Corporation

(21) Appl. No.: 11/741,123

(22) Filed: Apr. 27, 2007

Related U.S. Application Data
(60) Provisional application No. 60/909,286, filed on Mar. 30, 2007.

(51) Int. Cl. A61M 5/14

(52) U.S. Cl. 604/28

(57) ABSTRACT

A method for inserting a cannula of an infusion set into an infusion site includes releasably retaining a cannula cartridge of an infusion set on a distal end of a cannula insertion device plunger. The method also includes releasably retaining an infuser base on a distal end of a housing of the cannula insertion device. Subsequently, the method involves moving the plunger from a retracted position, wherein the cannula cartridge is remote from the infuser base, to an advanced position wherein the cannula cartridge is engaged with the infuser base and a cannula of the cannula cartridge has been subcutaneously inserted into an infusion site by a cannula insertion needle attached to the plunger.

### CROSS-REFERENCE

This application claims the benefit of U.S. Provisional Application No. 60/909,286, filed Mar. 30, 2007, which is incorporated herein by reference in its entirety.

### BACKGROUND OF THE INVENTION

1. Field of the Invention

The present invention relates, in general, to medical devices and, in particular, to infusion sets, cannula insertion devices, infusion set kits and related methods.

2. Description of the Related Art

Frequent or continuous subcutaneous injection of a medication (e.g., insulin) or other substance is often accomplished with the use of an infusion set. During use, such an infusion set is mounted to an infusion site on a user's skin with a cannula of the infusion set extending through the user's skin. A medication or other substance is then delivered to the infusion site through the cannula. The source of the medication can be, for example, a medication pump (such as an insulin pump) connected to the infusion set via a fluid line. U.S. Pat. No. 6,572,586, which is hereby incorporated in full by reference, includes descriptions of infusion sets and their various components.

Insertion devices are typically employed to mount an infusion set, or components thereof, on an infusion site and/or to extend an infusion set cannula through the user's skin. Such insertion devices frequently employ an insertion needle to place the cannula in a subcutaneous layer of the user's skin. Further details related to insertion devices are in U.S. Pat. No. 7,052,483, which is hereby incorporated in full by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings in which like numerals indicate like elements and of which:

FIG. 1 is a simplified perspective view of an infusion set according to an exemplary embodiment of the present invention with the adhesive pad thereof not shown for simplicity;

FIG. 2 is a simplified side view of the infusion set of FIG. 1;

FIG. 3 is a simplified perspective view of the infuser base (including adhesive pad) and cannula cartridge of the infusion set of FIG. 1;

FIG. 4 is a simplified perspective view of a portion of the infuser base of FIG. 3;

FIG. 5 is a simplified perspective view of the hub of the infusion set of FIG. 1 with the directions of 360-degree hub rotary motion indicated by a double-headed arrow;

FIG. 6 is a simplified perspective view of an infusion set according to another exemplary embodiment of the present invention;

FIG. 7 is a simplified side view of the infusion set of FIG. 6;

FIG. 8 is a simplified cross-sectional view of the infusion set of FIG. 6;

FIG. 9 is a simplified top view of the infuser base and cannula cartridge of the infusion set of FIG. 6;

FIG. 10 is a simplified perspective view of the infuser base and cannula cartridge of FIG. 9;

FIG. 11 is a simplified side view of the infuser base and cannula cartridge of FIG. 9;

FIG. 12 is a simplified bottom perspective view of the hub of the infusion set of FIG. 6;

FIG. 13 is a simplified perspective view of the cannula cartridge of the infusion set of FIG. 6;

FIG. 14 is a simplified cross-sectional view of the cannula cartridge of FIG. 13;

FIG. 15 is a simplified perspective view of an infusion set according to yet another exemplary embodiment of the present invention;

FIG. 16 is a simplified perspective view of the infuser base of the infusion set of FIG. 15;

FIG. 17 is a simplified bottom view of the infusion set of FIG. 15, absent the adhesive pad;

FIG. 18 is a bottom view of the hub of the infusion set of FIG. 15, absent the flexible line;

FIG. 19 is a simplified bottom view of a hub as can be employed in infusion sets according to various exemplary embodiments of the present invention;

FIG. 20 is a simplified perspective view of a infuser base (with the adhesive pad thereof not shown) as can be employed with the hub of FIG. 19 in infusion sets according to various exemplary embodiments of the present invention;

FIG. 21 is a flow diagram depicting stages in a process according to an exemplary embodiment of the present invention;

FIG. 22 is a simplified perspective view of an infusion set according to an additional exemplary embodiment of the present invention;

FIG. 23 is a simplified exploded perspective view of the infusion set of FIG. 22;

FIG. 24 is a simplified perspective view depicting the hub and tube set connector removed from the infuser base of the infusion set of FIG. 22;

FIG. 25 is a simplified perspective view depicting the tube set connector removed from the hub of the infusion set of FIG. 22;

FIG. 26 is a simplified cross-sectional view of the infuser base of the infusion set of FIG. 22;

FIG. 27 is a simplified cross-sectional view of the cannula cartridge of the infusion set of FIG. 22;

FIG. 28 is a simplified cross-sectional view of the infuser base and cannula cartridge of FIGS. 26 and 27 attached to an infusion site on a user's skin (SK);

FIG. 29 is a simplified cross-sectional view of the hub of the infusion set of FIG. 22;

FIG. 30 is a simplified cross-sectional view of the hub of FIG. 22 attached to the infuser base and cannula cartridge of FIG. 28;

FIG. 31 is a simplified cross-sectional view of the tube set connector of the infusion set of FIG. 22;

FIG. 32 is a simplified cross-sectional view of the tube set connector of FIG. 31 laterally attached to the hub, infuser base and cannula cartridge of FIG. 30 in a user-releasable manner with the arrows indicating directions of tube set connector attachment and release;

FIG. 33 is a simplified top cross-sectional view of the tube set connector, hub, infuser base and cannula cartridge of FIG. 31;

FIG. 34 is a flow diagram depicting stages in a process according to another exemplary embodiment of the present invention;

FIG. 35 is a simplified side view of a cannula insertion device according to an exemplary embodiment of the present invention;

FIG. 36 is a simplified top perspective view of the cannula insertion device of FIG. 35;

FIG. 37 is a bottom perspective view of the cannula insertion device of FIG. 35 depicting a cannula cartridge attached to the cannula insertion device;

FIG. 38 is a simplified cross-sectional view of the cannula insertion device of FIG. 37 depicting a cannula cartridge and infuser base attached to the cannula insertion device;

FIG. 39 is a simplified side view of a cannula insertion device according to another exemplary embodiment of the present invention in a retracted state;

FIG. 40 is a simplified perspective view of the cannula insertion device of FIG. 39;

FIG. 41 is a simplified cross-sectional view of the cannula insertion device of FIG. 39;

FIG. 42 is a simplified perspective view of the housing of the cannula insertion device of FIG. 39;

FIG. 43 is a simplified perspective view of the plunger of the cannula insertion device of FIG. 39;

FIG. 44 is a simplified side view of the cannula insertion device of FIG. 39 in an advanced state;

FIG. 45 is a simplified cross-sectional side view of the cannula insertion device of FIG. 44;

FIG. 46 is a simplified cross-sectional view of an automatic insertion module in use with the cannula insertion device of FIGS. 39 through 45 absent a cannula insertion device cap;

FIG. 47 is a simplified perspective view of a cannula insertion device according to yet another exemplary embodiment of the present invention in a retracted state;

FIG. 48 is a simplified cross-sectional view of the cannula insertion device of FIG. 47;

FIG. 49 is a simplified exploded side view of the cannula insertion device of FIG. 47;

FIG. 50 is a simplified perspective view of the cannula insertion device of FIG. 47 in an advanced state;

FIGS. 51A and 51B are simplified cross-sectional views of the cannula insertion device of FIG. 50;

FIG. 52 is a simplified cross-sectional view of a cannula insertion device according to an additional exemplary embodiment of the present invention; and

FIG. 53 is a flow diagram depicting stages in process according to yet another exemplary embodiment of the present invention.

### DETAILED DESCRIPTION OF ILLUSTRATIVE EMBODIMENTS OF THE INVENTION

While preferred embodiments of the present invention are shown and described herein, it will be apparent to those skilled in the art that such embodiments are provided by way of example only. Numerous variations, changes, and substitutions will now occur to those skilled in the art without departing from the invention.

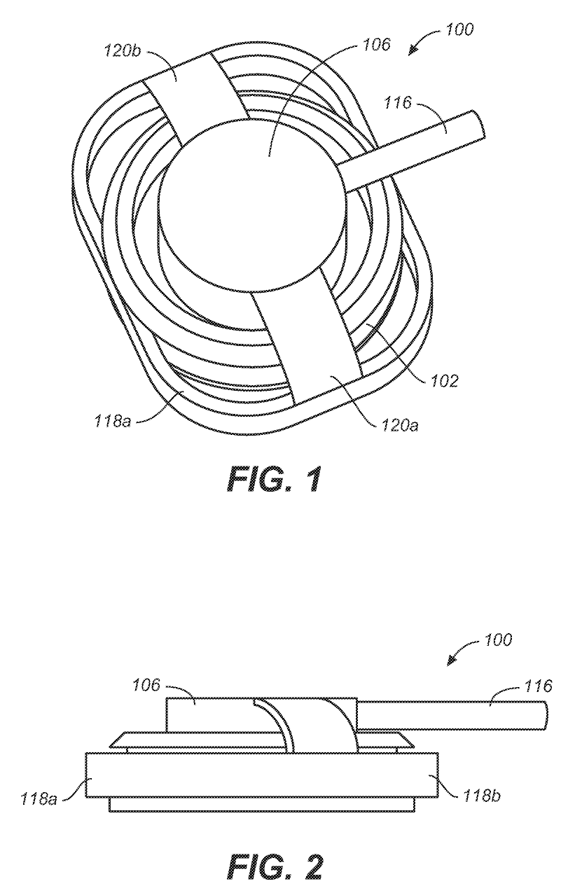

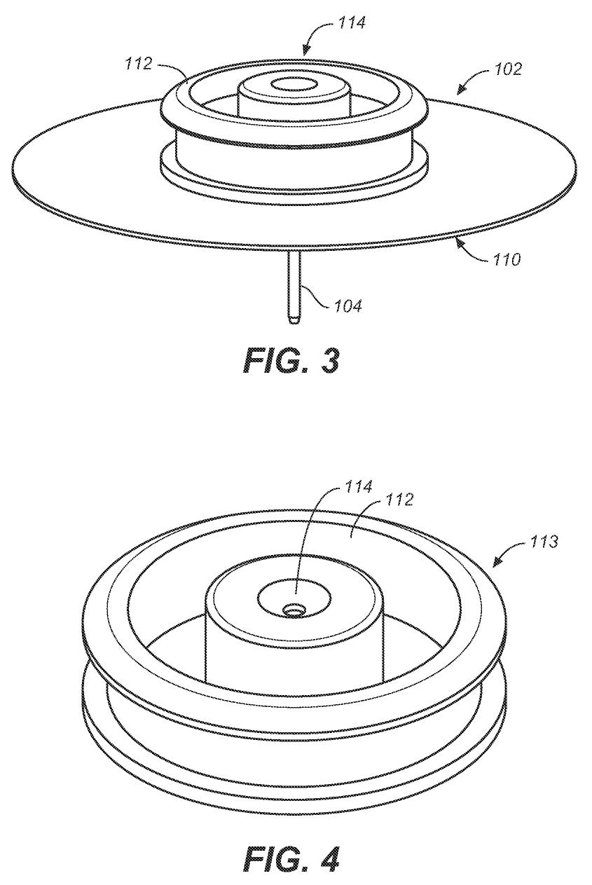

FIGS. 1 through 5 are various depictions of an infusion set 100 according to an embodiment of the present invention. Referring to FIGS. 1-5, infusion set 100 includes an infuser base 102, a cannula cartridge 104 (of which only a cannula is visible in FIG. 3), and a hub 106. It is contemplated that infusion set 100 can be supplied to a user in a sterile package (not shown in FIGS. 1-5)

Infuser base 102 includes an adhesive pad 110 (shown in FIG. 3 only) for removably adhering infuser base 102 to an infusion site on a user's skin, a barb 112 and an infuser base opening 114 that extends through infuser base 102. Infuser base 102 can be made of any suitable material including, for example, suitable clear plastic materials that provide visibility of the infusion site to a user.

Barb 112 of infuser base 102 is generally circular and symmetric about a center axis of infuser base 102. Barb 112 has a uni-directional tapered edge 113 to assist in alignment of barb 112 and hub 106 during the attachment of hub 106 to infuser base 102. Moreover, infuser base opening 114 is also tapered (as is most evident in FIG. 4) to serve as a guide for insertion of cannula cartridge 104 into infuser base 102.

Cannula cartridge 104 includes a cannula (depicted in FIG. 3) for subcutaneous insertion into an infusion site and a cannula cartridge self-sealing septum (not depicted in FIGS. 1-5, but further described herein with respect to other infusion set embodiments of the present invention).

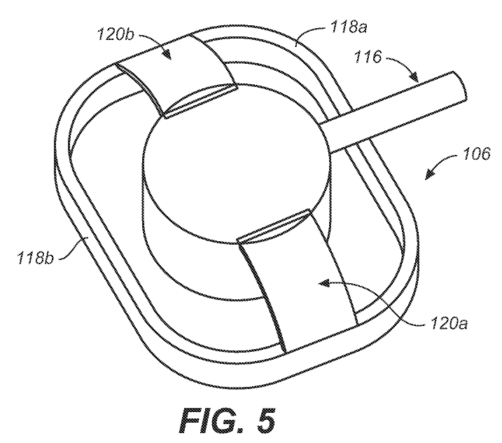

Hub 106 is configured for removable and user-controlled three hundred and sixty degree (360°) rotateable engagement with barb 112 of the infuser base 102. Therefore, hub 106 is also referred to as a 360° user-controlled rotary motion hub. The direction of rotary motion is shown by a double-headed arrow in FIG. 5 and is in a plane essentially parallel to the user's skin when infusion set 100 is mounted on an infusion site. Hub 106 also includes a hub needle (not shown in FIGS. 1-5) for piercing the self-sealing septum of cannula cartridge 104, thereby creating a fluid pathway from the hub needle to the cannula of cannula cartridge 104. Hub 106 also includes a flexible fluid line 116 in fluid communication with the hub needle. One skilled in the art will recognize that flexible fluid line 116 can be connected to a source of medication (such as an insulin pump) or other substance that is to be frequently or continuously supplied to the infusion site via infusion set 100.

Hub 106 further includes resilient bands 118a and 118b configured for engagement with barb 112. Hub 106 also includes torsion bars 120a and 120b. When in a relaxed condition (as depicted in FIG. 5), resilient bands 118a and 118b are separated by a gap that is insufficient to allow attachment of hub 106 to barb 112. The application of pressure to torsion bars 120a and 120b by a user causes torsion bars 120a and 120b to deflect and resilient bands 118a and 118b to spread apart such that resilient bands 118a and 118b will pass across uni-directional tapered edge 113 and attach to barb 112 (see, for example, FIG. 1) via a gripping force provided by the resilient nature of resilient bands 118a and 116b.

It should be noted that pressure can be applied to torsion bars 120a and 120b in an indirect manner. For example, a user can roughly align hub 106 with infuser base 102 and press down on the center of hub 106, thus applying pressure to torsion bars 120a and 120b. In addition, resilient bands 118a and 118b and torsion bars 120a and 120b can be made of any suitable material (and thickness thereof) that will flex under load, return to a relaxed state when the load is removed, and provide a desired gripping force. Suitable materials include, but are not limited to, resilient polymers such as polypropylene, polycarbonate, polyurethane, polyethylene and combinations thereof.

During this attachment, the hub needle of hub 106 is automatically aligned with and inserted into infuser base opening 114 such that the self-sealing septum of the cannula cartridge 104 is pierced by the hub needle, thus creating a sealed fluid communication pathway from flexible fluid line 116 to the cannula of the cannula cartridge 104 for the delivery of medication or other substances to an infusion site.

Upon the release of pressure by a user, resilient bands 118a and 118b return to their original configuration and attach to (i.e., grip) barb 112 in a manner that allows manual 360 degree (360°) rotation of hub 106 by a user. Therefore, infusion set 100 has a user-controlled 360° rotary motion hub. This enables a user to position (and reposition) hub 106, including flexible fluid line 116, throughout 360° of rotation to increase comfort to the user or to provide a rotary position of the hub and flexible fluid line that most readily accommodates a user's activities. Moreover, the user-controlled rotary motion occurs without removing hub 106 from infuser base 102 and, therefore, without interrupting the delivery of medication or other substance to the infusion site. For example, the user may rotate the hub to facilitate repositioning an insulin pump without disconnecting the insulin pump from the infusion set. Additionally, it may be advantageous for a user to rotate the hub so that the flexible fluid line exits the infusion site at a suitable angle that prevents the flexible fluid line from becoming entangled. In addition, depending on the gripping force between resilient bands 118a and 118b and barb 112, beneficial rotary motion of hub 106 can also occur as a result of torque (force) applied to hub 106 by movement of flexible fluid line 116. The minimum force required to rotate hub 106 is, for example, in the range of 0.05 lbf to 0.1 lbf (applied essentially tangentially to the outer circumference of hub 106). Therefore, forces less than the minimum force that are inadvertently applied to hub 106 from, for example, the unsupported weight of flexible fluid line 116, will not result in unwanted rotary motion of hub 106. Once apprised of the present disclosure, one skilled in the art will recognize that the minimum force required to rotate hubs according to embodiments of the present invention can be predetermined by adjusting the aforementioned gripping force and can be any suitable minimum force.

A user can remove hub 106 from infuser base 102 by applying pressure to the torsion bars 120a and 120b, thereby deflecting (flexing) resilient bands 118a and 118b sufficiently that they can pass around generally circular barb 112, thereby disengaging (i.e., removing) hub 106 from infuser base 102.

In infusion set 100, cannula cartridge 104 is configured for insertion into infuser base opening 114 with the cannula being subcutaneously inserted into the infusion site essentially perpendicular to the insertion site user's skin (see, for example, FIG. 3 wherein cannula 104 is disposed along the center axis of infuser base 102 to provide for perpendicular subcutaneous insertion). Such a perpendicular insertion is referred to as a “straight” insertion and, therefore, infusion set 100 and other embodiments with a perpendicular cannula insertion are sometimes referred to as straight insertion infusion sets.

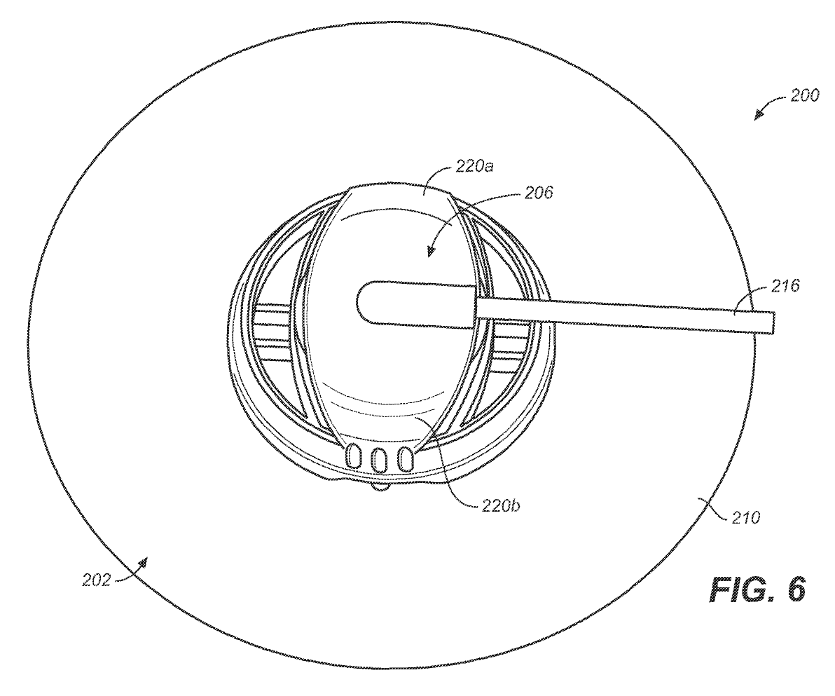

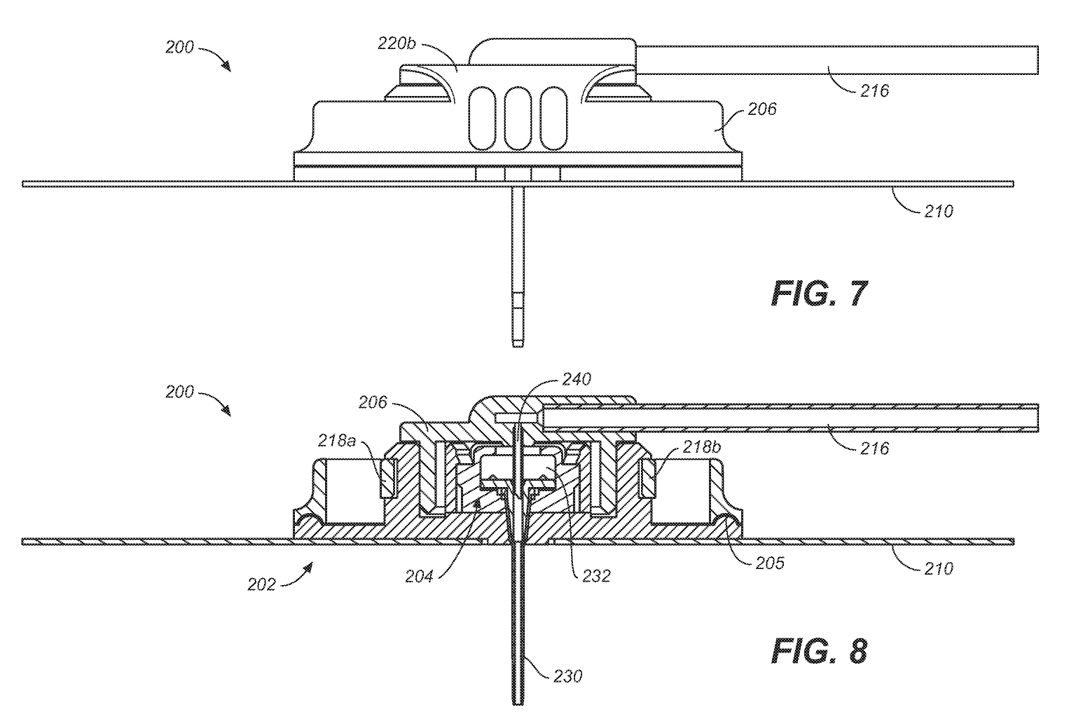

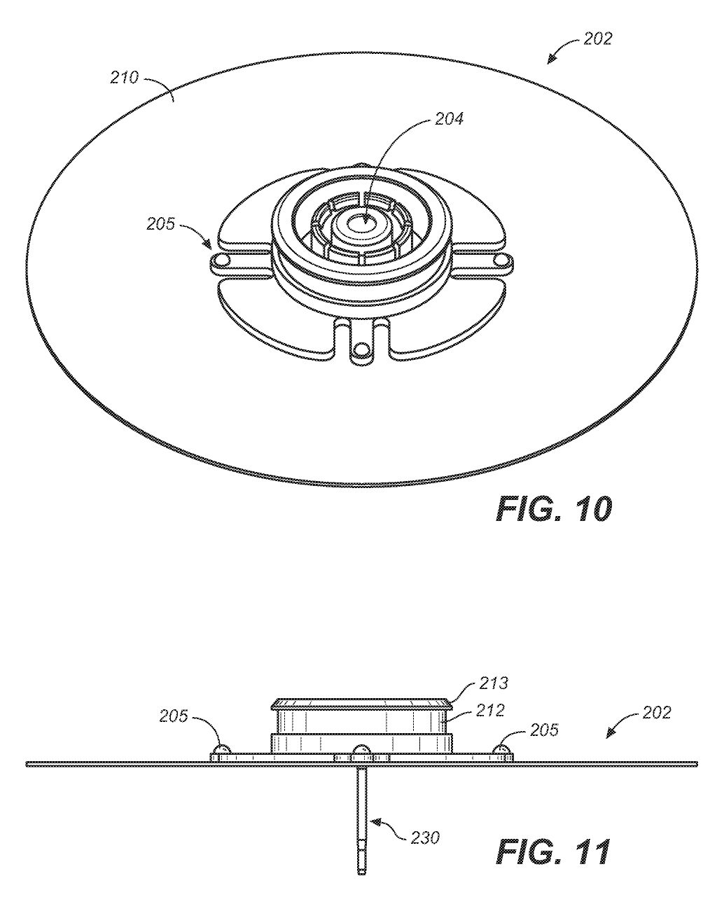

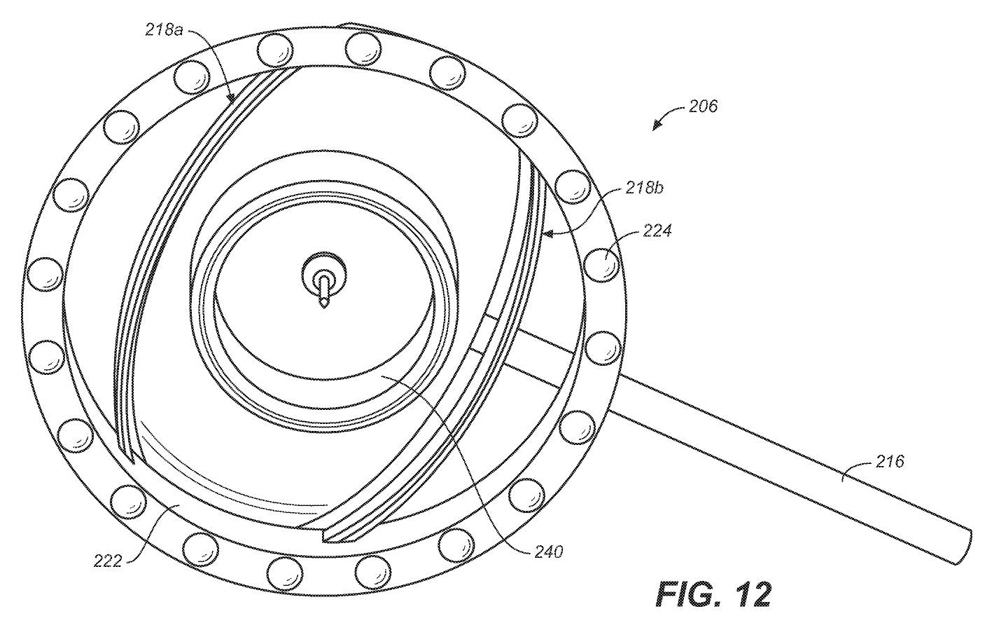

FIGS. 6 through 14 are various depictions of an infusion set 200 according to another exemplary embodiment of the present invention. Referring to FIGS. 6 through 14, infusion set 200 includes an infuser base 202, a cannula cartridge 204, and a hub 206.

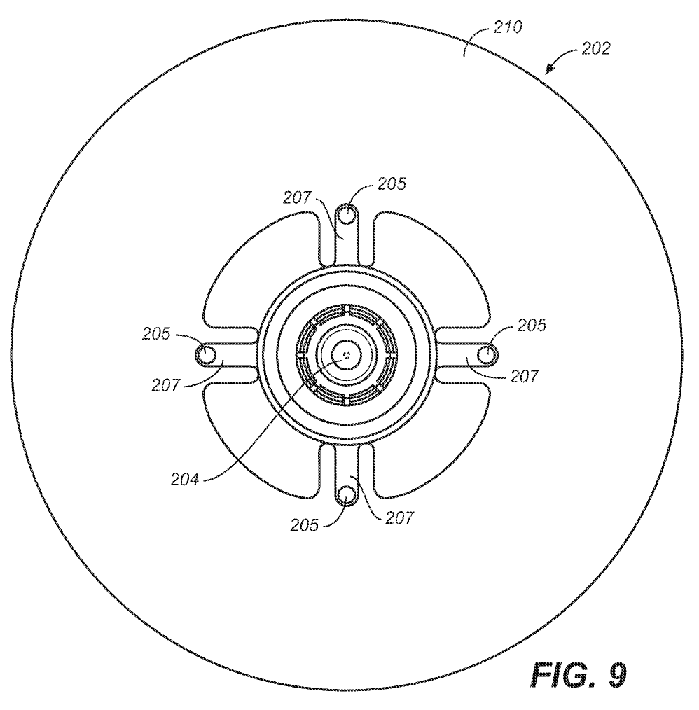

Infuser base 202 includes an adhesive pad 210 for removably adhering infuser base 202 to an infusion site on a user's skin, a barb 212 and an infuser base opening that extends through infuser base 202 and accommodates cannula cartridge 204 (as shown, for example, in FIG. 8). Infuser base 202 also includes a plurality of projections 205 with each projection being disposed on a leaf-spring arm 207 (see, in particular, FIG. 9). Infuser base 202 can be made of any suitable material including, for example, suitable clear plastic materials that provide visibility of the infusion site to a user. Although, for the purpose of illustration only, the projections are depicted as hemispherical in shape, any suitably shaped projections can be employed. Leaf-spring arm 207 is configured to deflect under pressure and provide a predetermined resistance to rotary motion as described below.

Barb 212 of infuser base 202 is generally circular and symmetric about a center axis of infuser base 202. Barb 212 has a uni-directional tapered edge 213 to assist in alignment during the attachment of hub 206 and to assist in the retention of hub 206 following attachment of hub 206 to infuser base 202.

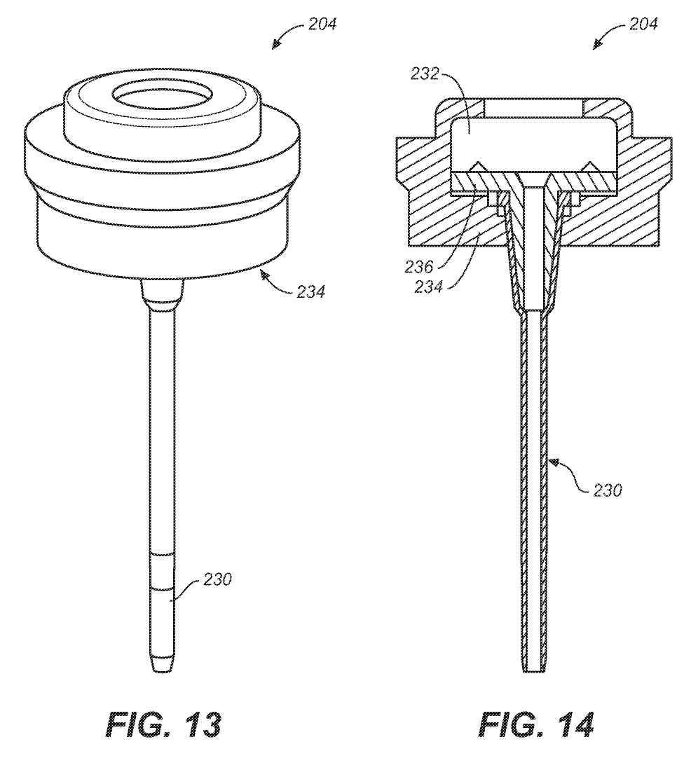

Cannula cartridge 204 includes a cannula 230 for subcutaneous insertion into an infusion site, a cannula cartridge self-sealing septum 232 (such as a silicone self-sealing septum), a cannula housing 234 and a cannula ferrule 236 (see, in particular, FIGS. 13 and 14). Cannula 230 can be formed of any suitable material, such as a flexible polymer (e.g., a Teflon flexible polymer). Moreover, if desired, cannula 230 can be formed of a super-elastic memory shape alloy such as NiTiNOL. NiTiNOL is particularly beneficial in that it can be used to form kink-resistant cannulas of relatively thin wall thickness and that are also sterilizable with radiation.

Hub 206 is configured for removable and user-controlled three-hundred and sixty degree (360°) rotateable engagement with barb 212 of infuser base 202. Hub 206 also includes a hub needle 240 for piercing cannula cartridge self-sealing septum 232 of cannula cartridge 204 (see, for example, FIGS. 8 and 12), thereby creating a fluid pathway from hub needle 240 to cannula 230 (see FIG. 8 in particular). Hub 206 also includes a flexible fluid line 216 in fluid communication with hub needle 240.

Hub 206 further includes resilient bands 218a and 218b configured for engagement with barb 212. Hub 206 also includes torsion bars 220a and 220b and an outer ring 222 with a plurality (i.e., twenty in the embodiment of infusion set 200) of indentations 224.

When in a relaxed condition, resilient bands 218a and 218b are separated by a gap that is smaller than the width of uni-directional tapered edge 213 of barb 212. The application of pressure to torsion bars 220a and 220b by a user causes torsion bars 220a and 220b to deflect and resilient bands 218a and 218b to spread apart such that resilient bands 218a and 218b will pass across uni-directional tapered edge 213 and attach to barb 212 (see, for example, FIG. 8). Pressure can be applied to torsion bars 220a and 220b in an indirect manner. For example, a user can approximately align hub 206 with infuser base 202 and press down on the center of hub 206, thus applying indirect pressure to torsion bars 220a and 220b.

During this attachment, the hub needle 240 will be automatically aligned with and pierce cannula cartridge self-sealing septum 232, thus creating a sealed fluid communication pathway from flexible fluid line 216 to cannula 230 (see, for example, FIG. 8).

Upon the release of pressure by a user, resilient bands 218a and 218b return to their original configuration and grip barb 212 in a manner that allows manual 360° rotation of hub 206 by a user. Therefore, infusion set 200 has a user-controlled 360° rotary motion hub. This enables a user to position (and reposition) hub 206, including flexible fluid line 216, throughout 360° of rotation. During such rotation, projections 205 cooperate with indentations 224 to provide tactile rotation feedback to a user. During attachment and rotation of hub 206, leaf-spring arms 207 enable projections 205 to move slightly in response to downward pressure from indentations 224. Although, for the purpose of illustration only, the indentations are depicted as circular in shape, any suitably shaped indentations can be employed as long as they can cooperate with the projections. The cooperation (mating) of projections 205 and indentations 224 provides predetermined resistance to the rotary motion of hub 206 that is in addition to the resistance provided by gripping force between resilient bands 218a and 218b and barb 212. However, both of these forces can be overcome by a user during manual rotation of hub 206. If desired, projections 205 and indentations 224 can be formed of materials that result in an audible “click” sound as hub 206 is rotated and the projections move from cooperation with one indentation to cooperation with another indentation. Such a “click” provides audible feedback of rotation to a user.

A user can remove hub 206 from infuser base 202 by applying pressure to the torsion bars 220a and 220b in a direction perpendicular to the direction of cannula insertion, thereby deflecting resilient bands 218a and 218b sufficiently that they can pass around uni-directional tapered edge 213 of generally circular barb 212 to disengage (i.e., remove) hub 206 from infuser base 202.

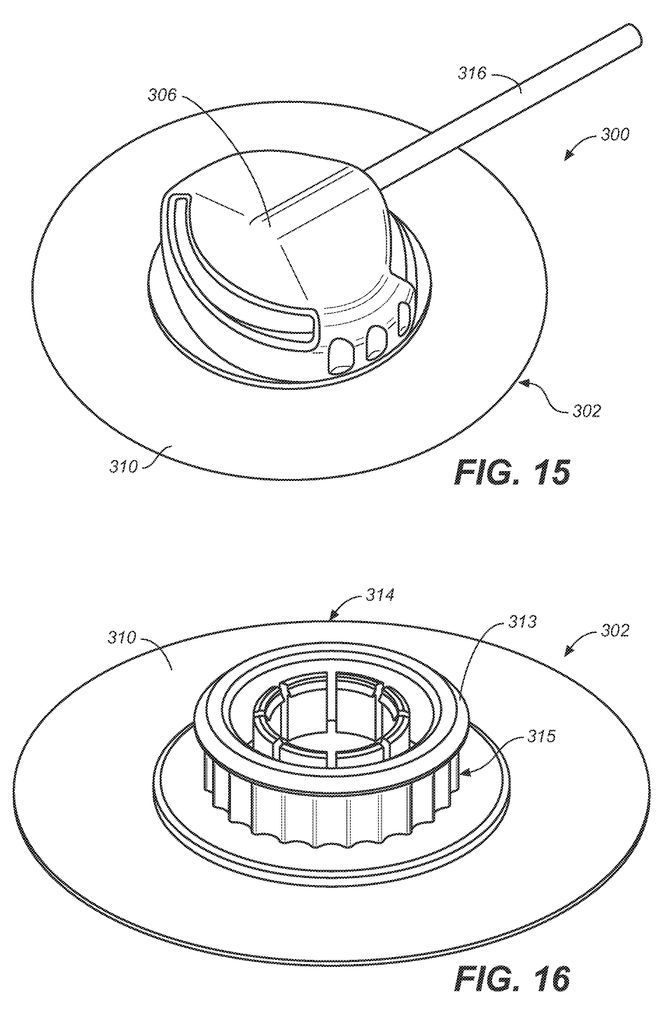

FIGS. 15 through 18 are various depictions of an infusion set 300 according to yet another exemplary embodiment of the present invention. Referring to FIGS. 15 through 18, infusion set 300 includes an infuser base 302, a cannula cartridge (not shown in FIGS. 15 through 18 but with features and characteristics as described in relation to other infusion set embodiments according to the present invention) and a hub 306.

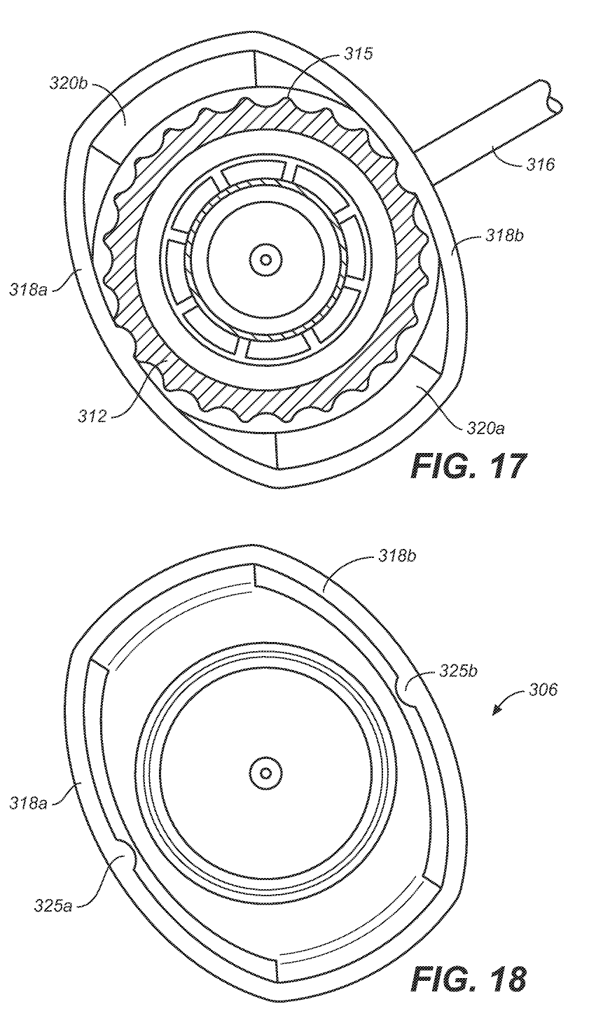

Infuser base 302 includes an adhesive pad 310 for removably adhering infuser base 302 to an infusion site on a user's skin, a barb 312 and an infuser base opening 314 that extends through infuser base 302 and accommodates the cannula cartridge. Infuser barb 312 includes a plurality of indentations 315 along a perimeter of barb 312 (see FIGS. 16 and 17).

Barb 312 of infuser base 302 is generally circular and symmetric about a center axis of infuser base 302. Barb 312 has a uni-directional tapered edge 313 to assist in alignment during the attachment of hub 306.

Hub 306 is configured for removable and user-controlled three hundred and sixty degree (360°) rotateable engagement with barb 312 of the infuser base 302. Hub 306 also includes a hub needle for piercing a self-sealing septum of the cannula cartridge (not depicted in FIGS. 15-18), thereby creating a fluid pathway from the hub needle to a cannula of the cannula cartridge. Hub 306 also includes a flexible fluid line 316 in fluid communication with the hub needle.

Hub 306 further includes resilient bands 318a and 318b configured for engagement with barb 312. Hub 306 also includes torsion bars 320a and 320b. Moreover, resilient bands 318a and 318b each include a projection (325a and 325b) configured for cooperation with the plurality of radially-spaced indentations 315 of barb 312. Projections and radially-spaced indentations can be in any suitable shape so long as they can cooperate with each other.

When in a relaxed condition, resilient bands 318a and 318b are separated by a gap that is smaller than the width uni-directional tapered edge 313 of barb 312. The application of pressure to torsion bars 320a and 320b by a user causes torsion bars 320a and 320b to deflect and resilient bands 318a and 318b to spread apart such that resilient bands 318a and 318b will pass across uni-directional tapered edge 313 and attach to barb 312 (see FIG. 17). For example, a user can align hub 306 with infuser base 302 and press down on the center of hub 306, thus applying pressure to torsion bars 320a and 320b.

Upon the release of pressure by a user, resilient bands 318a and 318b return to their original configuration and grip barb 312 in a manner that allows manual 360° rotation of hub 306 by a user. Therefore, infusion set 200 has a user-controlled 360° rotary motion hub. Once resilient bands 318a and 318b grip barb 312, projections 325a and 325b cooperate with indentations 315 to provide frictional force that enables a user to smoothly and accurately control the rotary position of hub 306. The resilient nature of resilient bands 318a and 318b provide a frictional force between the projections and indentations that securely maintains a given rotary position yet allow a user to overcome that force by manually rotating hub 306. The cooperation of projections 325a and 325b with indentations 315 also provide tactile feedback to a user on the rotation motion and position of hub 306.

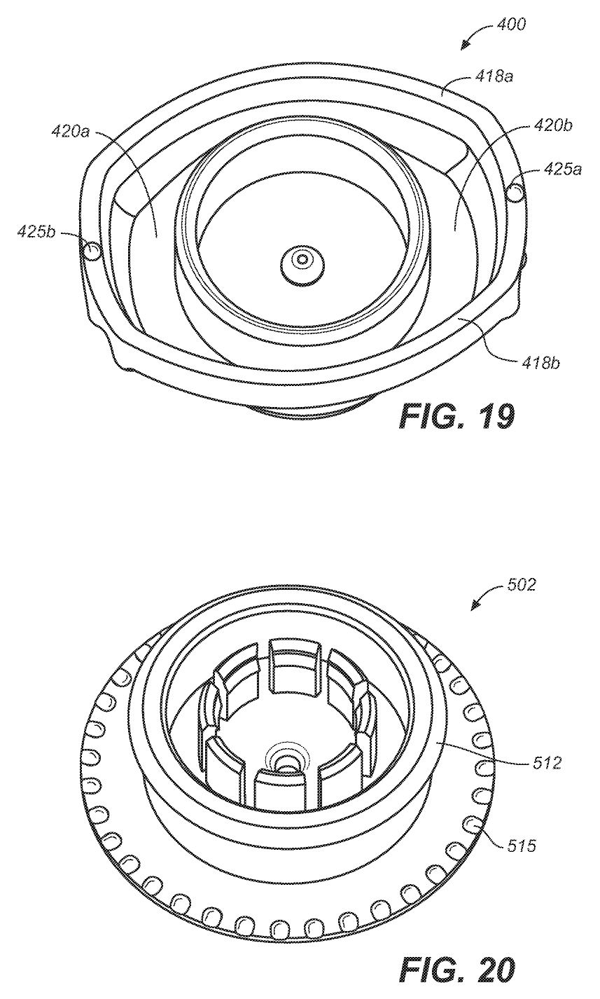

FIG. 19 is a depiction of a hub 400 that can be employed in infusion sets according to the present invention, while FIG. 20 is a depiction of an infuser base 500 as can also be employed in infusion sets according to the present invention along with hub 400.

Infuser base 500 includes an adhesive pad (not shown in FIG. 20) for removably adhering infuser base 500 to an infusion site on a user's skin, a barb 512 and a plurality of radially-spaced indentations 515 along a perimeter of barb 512.

Hub 400 is configured for removable and user-controlled three hundred and sixty degree (360°) rotateable engagement with barb 512 of the infuser base 502. Hub 400 further includes resilient bands 418a and 418b configured for engagement with barb 512. Hub 400 also includes torsion bars 420a and 420b. Moreover, resilient bands 418a and 418b each include a projection (425a and 425b) configured for cooperation with the plurality of radially-spaced indentations 515 of barb 512. Projections and radially-spaced indentations can be in any suitable shape so long as they can cooperate with each other.

Hub 400 and infuser base 500 cooperate such that projections 425a and 425b and radially-spaced indentations 515 securely maintain a given rotary position of hub 400 yet allow a user to manually control the rotary position by rotating hub 400.

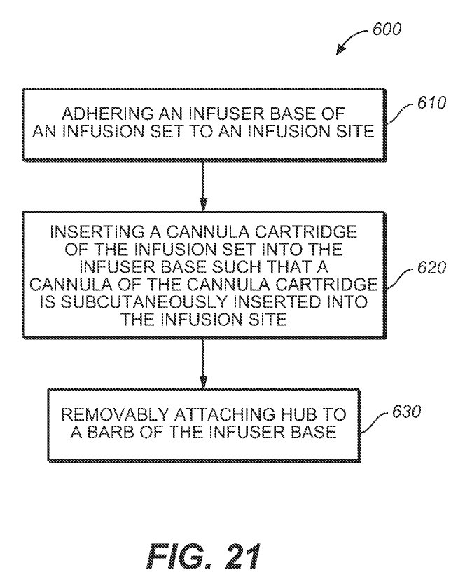

FIG. 21 is a flow diagram depicting stages in a method 600 for mounting an infusion set to an infusion site on a user's skin. Method 600 includes adhering an infuser base of the insertion set to an infusion site on a user's skin, as set forth in step 610.

At step 620 of method 600, a cannula cartridge of the infusion set is inserted into an opening of the infuser base such that a cannula of the cannula cartridge is subcutaneously inserted into the infusion site. Subsequently at step 630, method 600 includes removeably attaching a hub of the infusion set to a barb of the infuser base such that a hub needle of the hub pierces a self-sealing septum of the cannula cartridge, thereby creating a fluid pathway from the hub needle to the cannula. In method 600, the attachment of the hub to the barb is such that user-controlled three-hundred and sixty (360°) rotateable motion between the hub and the barb is provided.

Once apprised of the present disclosure, one skilled in the art will recognize that method 600 can be practiced using infusion sets according to embodiments of the present invention as well as cannula insertion devices according to embodiments of the present invention. Therefore, any of the functional characteristics and benefits described with respect to infusion sets, infusion kits and cannula insertion devices according to the present invention can be incorporated into method 600. Moreover, method 600 can, if desired, include a step of loading the cannula cartridge partially into the opening of the infuser base before the adhering step.

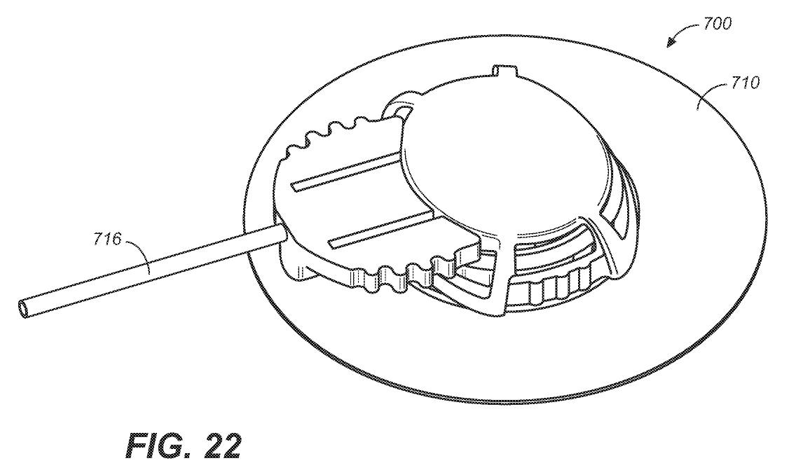

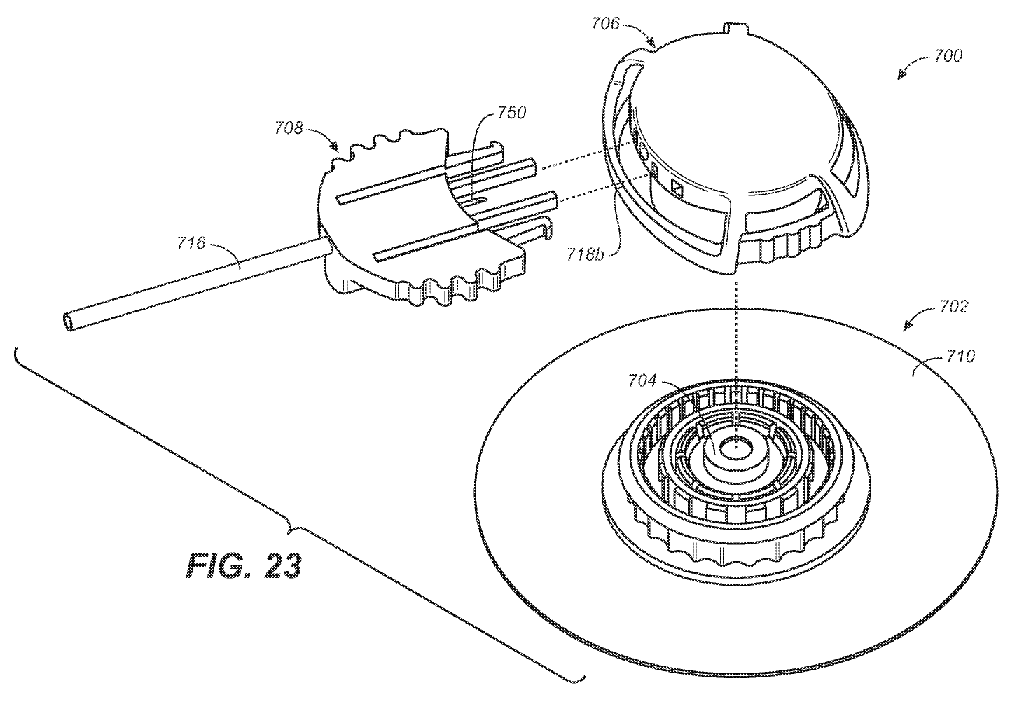

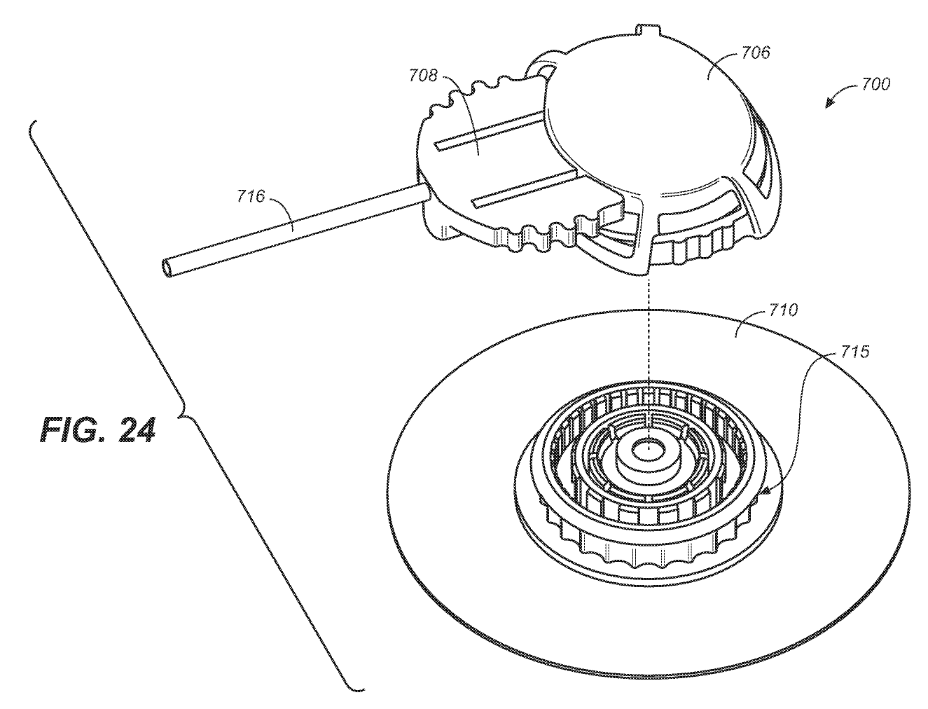

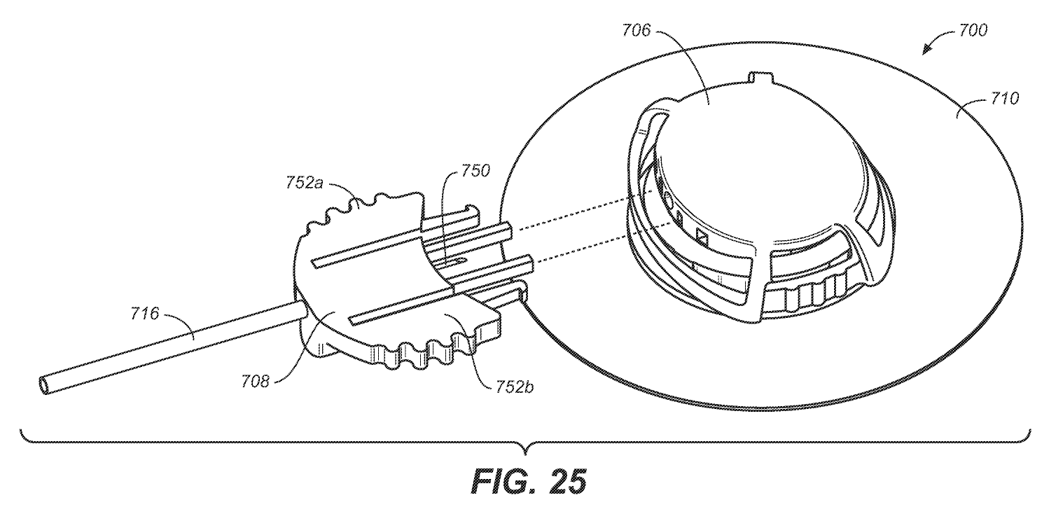

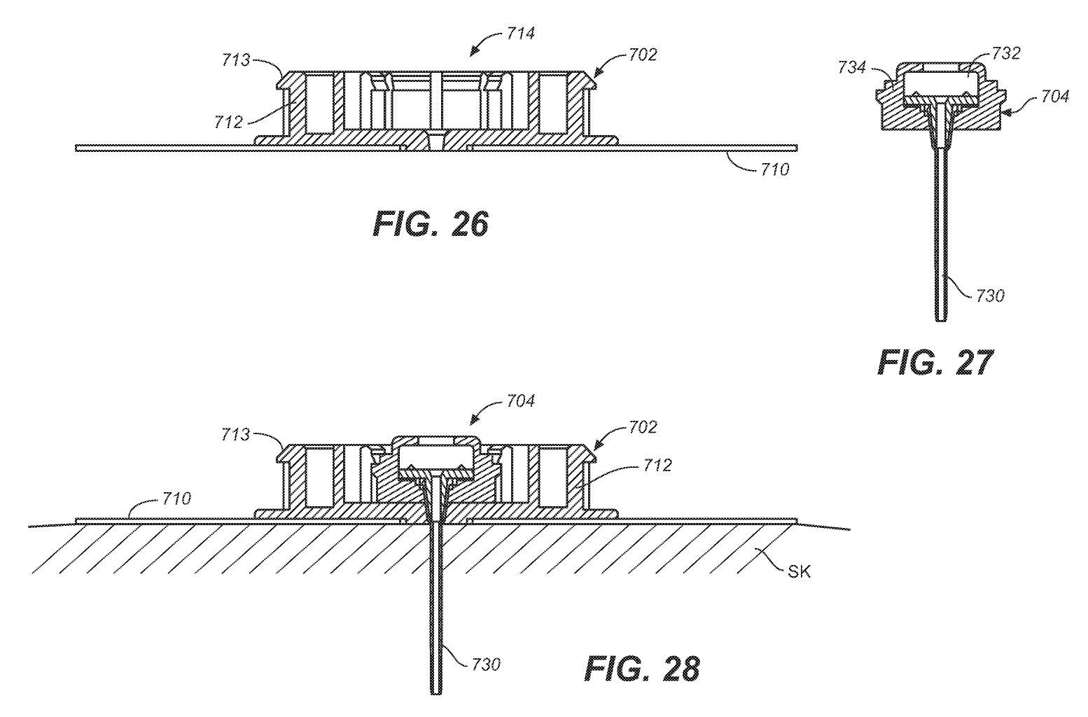

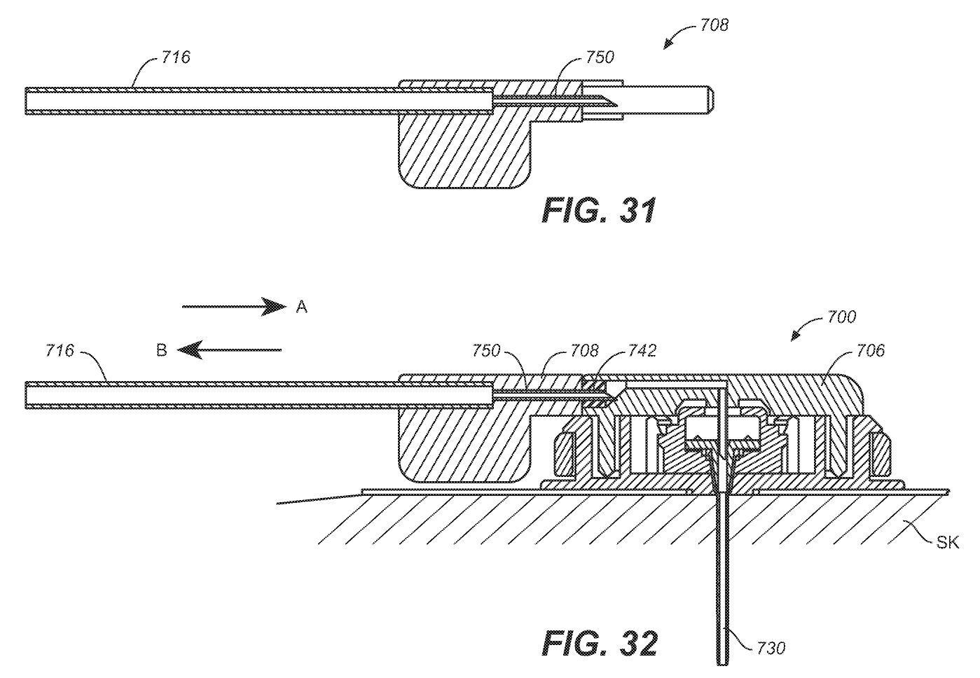

FIGS. 22 through 33 are various depictions of an infusion set 700 according to yet another exemplary embodiment of the present invention. Referring to FIGS. 22 through 33, infusion set 700 includes an infuser base 702, a cannula cartridge 704, a hub 706, and a tube set connector 708.

Infuser base 702 includes an adhesive pad 710 for removably adhering infuser base 702 to an infusion site on a user's skin (SK), a barb 712 and an infuser base opening 714 that extends through infuser base 702 and is accommodates cannula cartridge 704 (as shown, for example, in FIG. 28). Infuser base 702 also includes a plurality of radially-spaced indentations 715 along a perimeter of infuser base 702. Infuser base 702 can be made of any suitable material including, for example, suitable clear or tinted plastic materials (such as, for example, clear polycarbonate materials) that provide visibility of the infusion site to a user.

Barb 712 of infuser base 702 is generally circular and symmetric about a center axis of infuser base 702. Barb 712 has a uni-directional tapered edge 713 to assist in alignment during the attachment of hub 706.

Cannula cartridge 704 includes a cannula 730 for subcutaneous insertion into an infusion site, a cannula cartridge self-sealing septum 732, and a cannula housing 734.

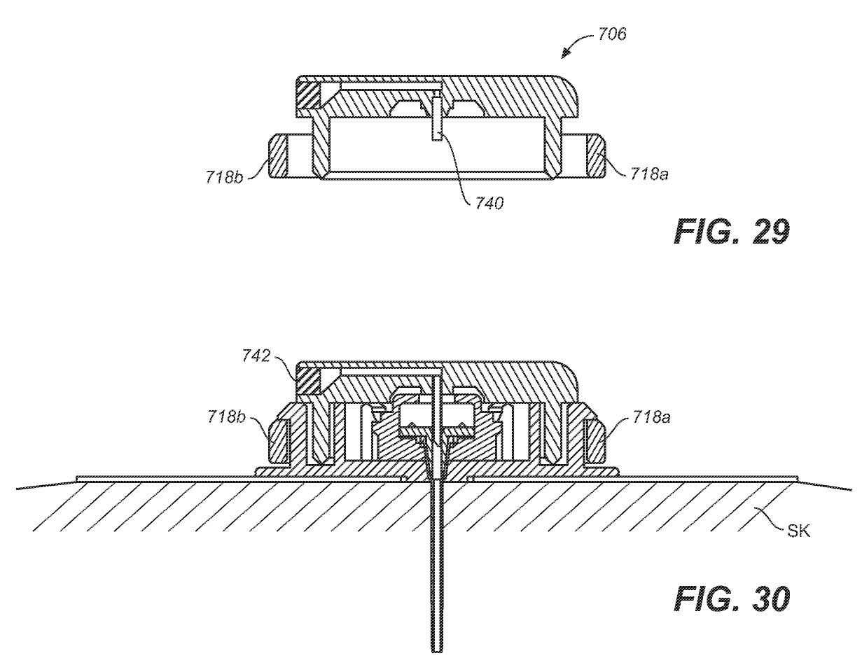

Hub 706 is configured for removable and user-controlled three hundred and sixty degree (360°) rotateable engagement with barb 712 of the infuser base 702. Hub 706 also includes a hub needle 740 for piercing the cannula cartridge self-sealing septum 732 of the cannula cartridge (see, for example, FIGS. 29 and 30), thereby creating a fluid pathway from hub needle 740 to cannula 730. Hub 706 also includes a hub self-sealing septum 742 (see, in particular, FIGS. 30 and 32).

Hub 706 further includes resilient bands 718a and 718b configured for engagement with barb 712. Hub 706 also includes torsion bars 720a and 720b. Resilient bands 718a and 718b also include a projection (not shown) configured for cooperation with the plurality of radially-spaced indentations 715 of barb 712.

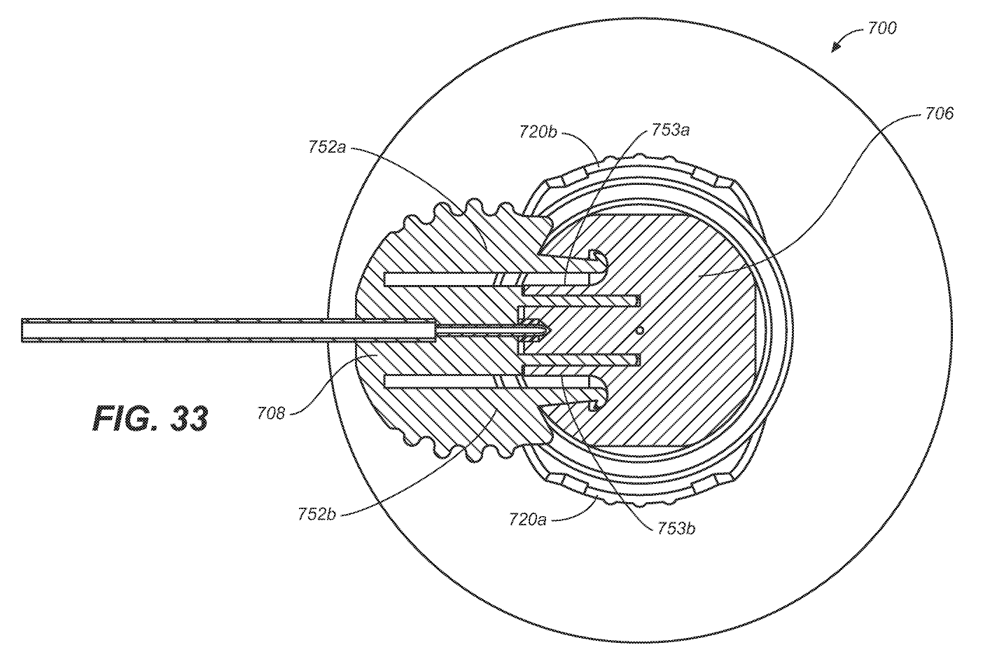

Tube set connector 708 is configured for user releasable lateral attachment to hub 706 and includes a tube set needle 750 for piercing hub self-sealing septum 742, thereby creating a fluid pathway from tube set connector 708 to the hub 706. Tube set connector 708 also includes a flexible fluid line 716 in fluid communication with tube set needle 750. Tube set connector 708 also includes two bendable members 752a and 752b (each configured similar to a leaf-spring) configured for providing the aforementioned releasable lateral attachment via latching. Bendable members 752a and 752b are configured for sliding insertion into, and to releasably mate with, openings 753a and 753b of hub 706 during lateral attachment of tube set connector 708 to hub 706 (see FIG. 33 in particular). Moreover, when bendable members 752a and 752b are subsequently squeezed together by a user with an applied force of, for example, approximately 0.25 lbf, bendable members 752a and 752b disengage from openings 753a and 753b by deflecting (i.e., bending) inward, thereby releasing tube set connector 708 from hub 706. Once apprised of the present disclosure, one skilled in the art will recognize that means for attaching a tube set connector to a hub in a user releasable lateral manner other than the bendable members described above can be employed in embodiments of the present invention. For example, suitable spring-based latches, spring-based detent pins and/or leaf springs can be employed.

The user-releasable lateral attachment of tube set connector 708 occurs in a direction that is perpendicular to the direction of cannula insertion into the infusion site (see, for example, FIG. 32 where arrow A indicates the direction of attachment and arrow B the direction of release). This “lateral” attachment and release (also referred to as side-attach) is beneficial in that attachment and release occurs without applying a force along the axis (direction) of cannula insertion. This reduces the opportunity for cannula separation from the infusion site during release, and such attachment is also believed to reduce force-related adhesive pad failure. Moreover, for users that experience pain upon the application of axial pressure to an infusion site, lateral attachment can be beneficial in avoiding such pain.

The function and characteristics of resilient bands (such as resilient bands 718a and 718b), torsion bars (such as torsion bars 720a and 720b), indentations (such as radially-spaced indentations 715) and projections have been described with respect to previous embodiments of infusion sets according to the present invention. Therefore, once apprised of the present disclosure, one skilled in the art will readily recognize that the resilient bands and torsion bars (as well as the indentations and projections) of infusion set 700 function in a similar manner to provide user-controlled 360° rotary motion for hub 706.

A benefit of infusion sets according to the present invention is that they include a separate infuser base and cannula cartridge. Therefore, a user can verify that the infuser base is correctly adhered to an infusion site before inserting the cannula cartridge into the infuser base and, thus, before the cannula of the cannula cartridge is subcutaneously inserted into the infusion site. By verifying that the infuser base is correctly adhered before inserting the cannula cartridge into the infuser base, the risk of improper cannula subcutaneous insertion will be reduced.

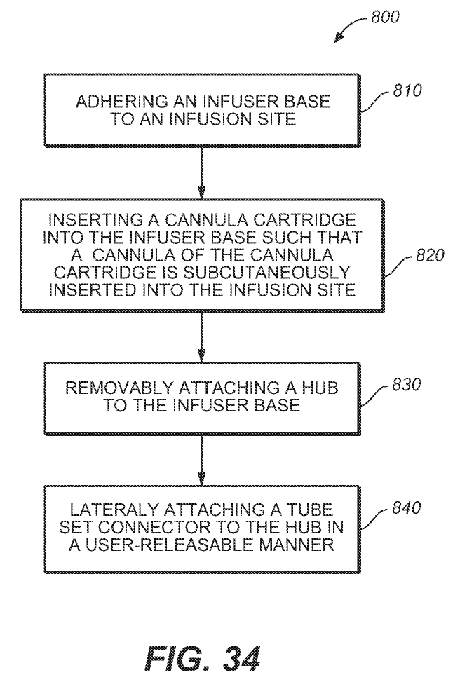

FIG. 34 is a flow diagram depicting stages in a method 800 for mounting an infusion set to an infusion site. Method 800 includes adhering an infuser base of the insertion set to an infusion site, as set forth in step 810.

At step 820, a cannula cartridge of the infusion set is inserted into an opening of the infuser base such that a cannula of the cannula cartridge is subcutaneously inserted into the infusion site. Method also includes subsequently removeably attaching a hub of the infusion set to a barb of the infuser base such that a hub needle of the hub pierces a self-sealing septum of the cannula cartridge, thus creating a fluid pathway from the hub needle to the cannula (see step 830). Further included at step 840 of method 800 is laterally attaching a tube set connector to the hub, in a direction perpendicular to the direction of cannula insertion, in a user-releasable manner, thus mounting the infusion set to the infusion site. In the method, the attachment of the hub to the barb is such that it provides for user-controlled three-hundred and sixty (360°) rotateable engagement between the hub and the barb. Means for achieving such rotateable engagement have been described herein with respect to infusion sets according to the present invention.

Once apprised of the present disclosure, one skilled in the art will recognize that method 800 can be practiced using infusion sets according to embodiments of the present invention as well as cannula insertion devices according to embodiments of the present invention. Therefore, functional characteristics and benefits described with respect to infusion sets, infusion kits and cannula insertion devices according to the present invention can be incorporated into method 800. Moreover, method 800 can, if desired, include a step of loading the cannula cartridge partially into the opening of the infuser base before the adhering step.

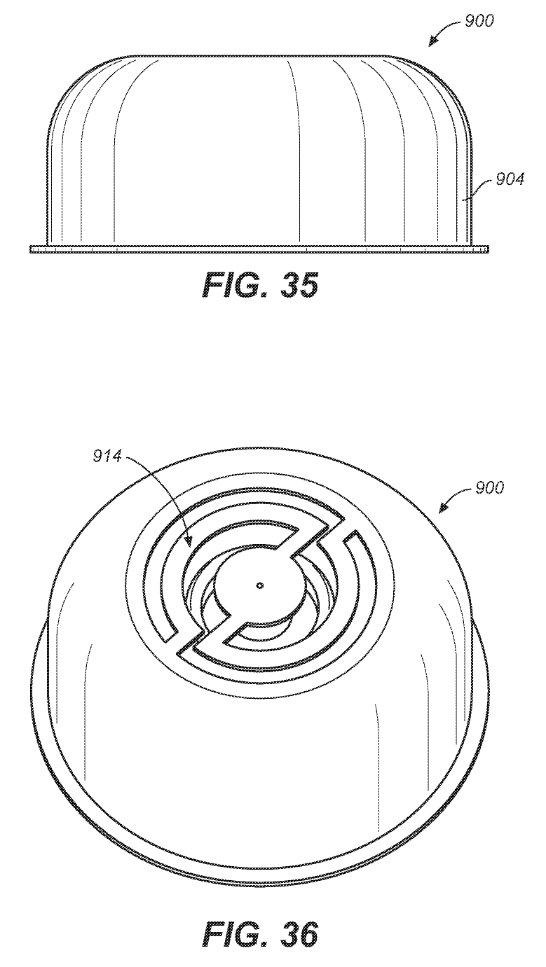

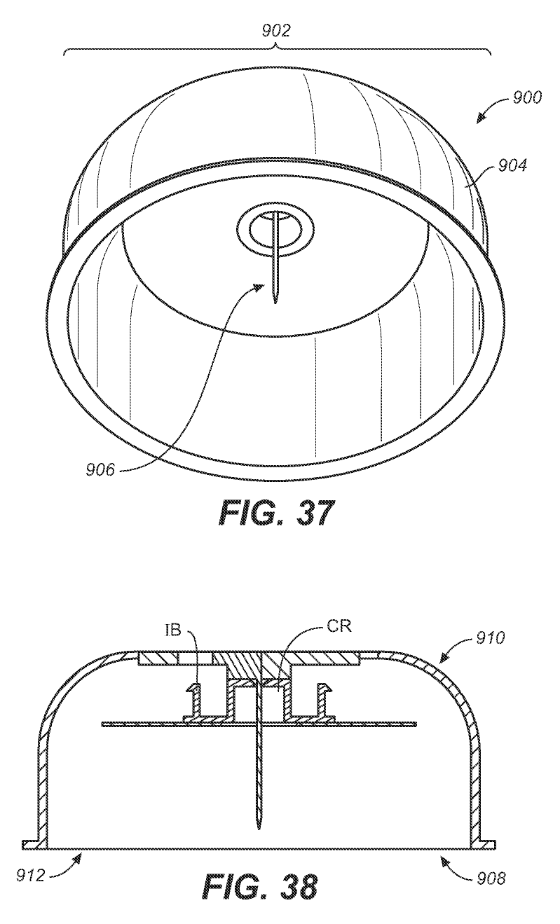

FIGS. 35-38 are various depictions of a cannula insertion device 900 for use with an insertion set according to an exemplary embodiment of the present invention. Referring to FIGS. 35-38, cannula insertion device 900 includes an insertion module 902 (with a housing 904) and a cannula insertion needle 906. It is envisioned that cannula insertion device 900 would be provided to a user as a sealed sterile package pre-loaded with a cannula cartridge CR and infuser base IB. Although, for the purpose of illustration only, the housing is depicted as dome-shaped, any suitably shaped housing can be employed. Moreover, the housing can be made of any suitable material, including suitable rigid and flexible materials.

The housing includes a housing distal end 908, a housing proximal end 910, a housing opening 912 and a torsion bar 914 integrated into the housing proximal end 910. Moreover, cannula insertion needle 906 is attached to torsion bar 914.

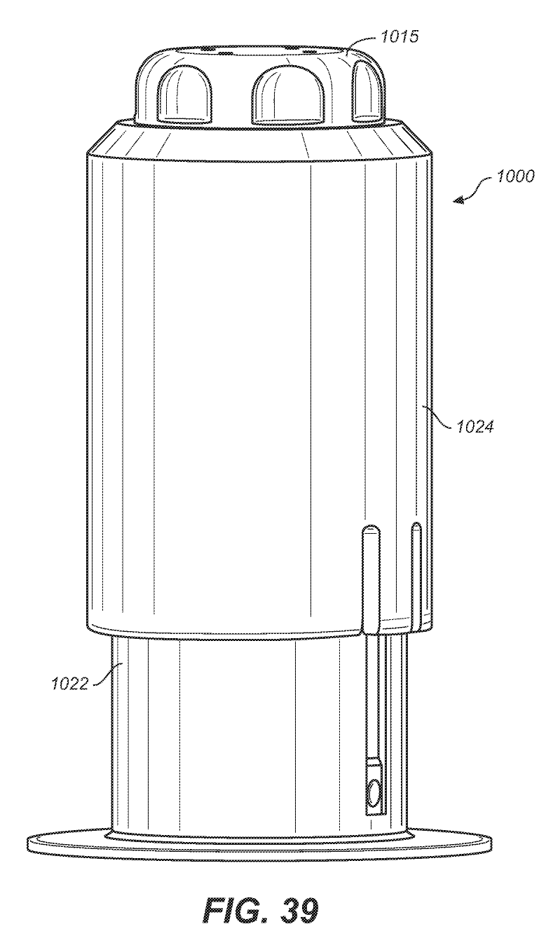

Referring to FIG. 38 in particular, torsion bar 914 is configured to releasably retain a cannula cartridge CR and an infuser base IB of the infusion set in operative alignment with cannula insertion needle 906. The cannula cartridge CR and infuser base IB can, for example, be releasably retained via frictional engagement with cannula insertion needle 906. Release of the cannula cartridge CR and infuser base IB from torsion bar 914 readily occurs following adhesive attachment of the infuser base IB to an infusion site since such adhesive attachment is stronger than the force of the aforementioned frictional engagement.

Torsion bar 914 is moveable from a retracted position, wherein a cannula cartridge and an infuser base releasably retained on torsion bar 914 are completely within housing opening 912 (as depicted in FIG. 38) and an advanced position wherein a cannula of a cannula cartridge has been subcutaneously inserted into an infusion site by the cannula insertion needle (a position not shown in FIGS. 35 through 38).

During use, a user would place housing distal end 908 against an infusion site and apply pressure to torsion bar 914 causing cannula insertion needle 906, cannula cartridge CR and infuser base IB to be advanced to the infusion site and causing a cannula of cannula cartridge CR to be subcutaneously inserted into the infusion site by action of cannula insertion needle 906. Moreover, advancement of infuser base IB to the infusion site also causes infuser base IB to be adhesively attached to the infusion site). Once the user removes the pressure applied to torsion bar 914, cannula insertion needle 906 would be automatically withdrawn (retracted) by the spring force of torsion bar 914.

It should be noted that the housing 904 (be it dome-shaped or any other suitable shape) serves to protect a user from accidentally encountering cannula insertion needle 906.

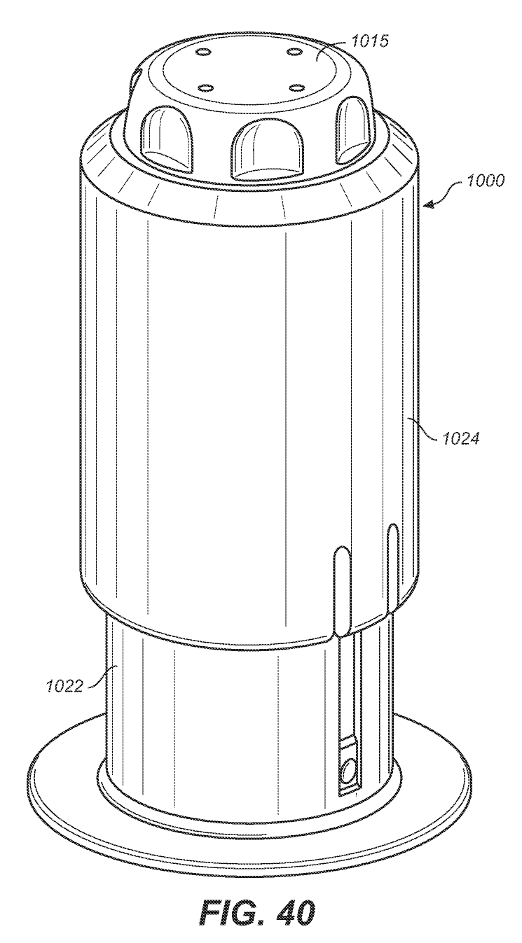

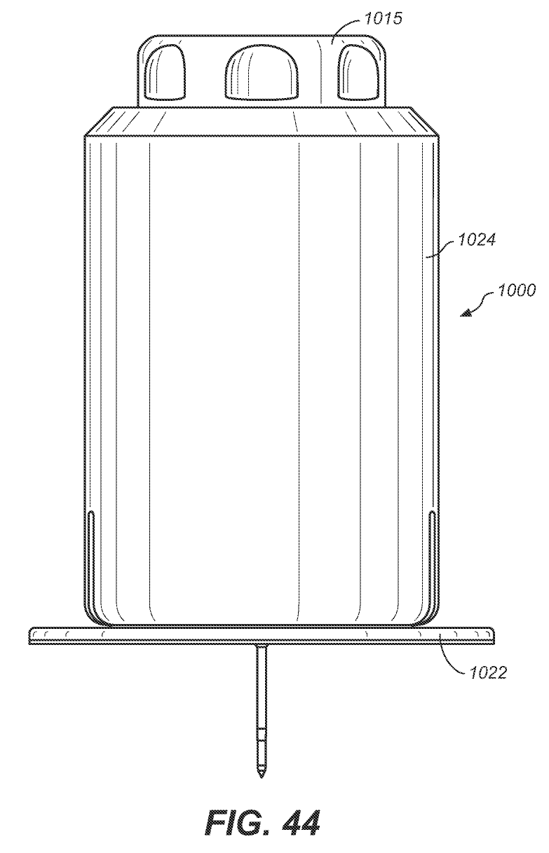

FIGS. 39 through 45 are various depictions of a cannula insertion device 1000, for use with an insertion set that includes an infuser base and cannula cartridge, according to another exemplary embodiment of the present invention. Cannula insertion device 1000 includes an insertion module 1010, a cap 1015 and a cannula insertion needle 1020.

Insertion module 1010 includes a housing 1022 and a plunger 1024. Housing 1022 has a housing distal end 1025, a housing proximal end 1026 and a housing opening 1028 extending from housing distal end 1025 to the housing proximal end 1026.

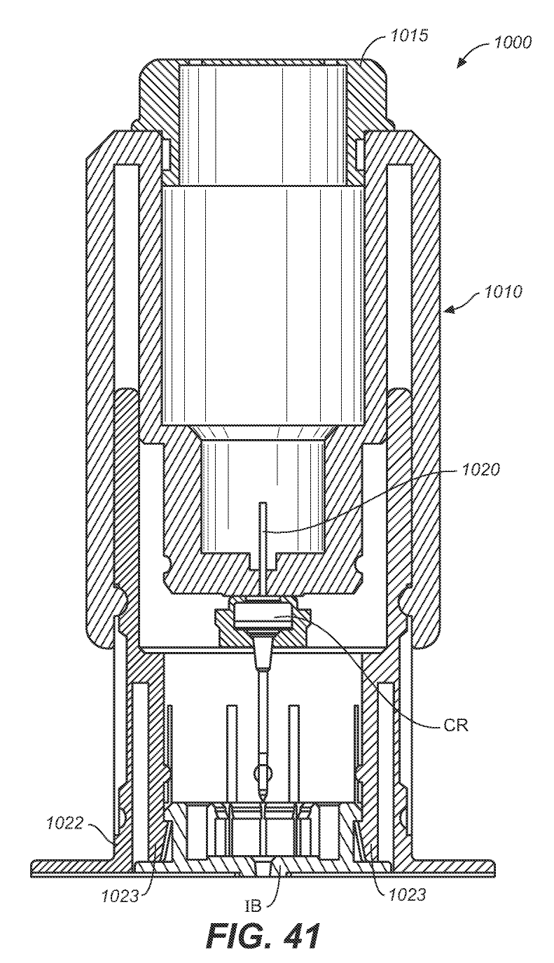

Plunger 1024 is disposed at least partially within housing opening 1028. Moreover, plunger 1024 includes a plunger distal end 1030 and a plunger proximal end 1032. Cannula insertion needle 1020 is attached to plunger distal end 1030 (see, for example, FIG. 41).

The distal end of the plunger is configured to releasably retain a cannula cartridge CR of the infusion set in operative alignment with cannula insertion needle 1020. The housing distal end is configured to releasably retain an infuser base IB of the infusion set in operative alignment with the cannula cartridge CR.

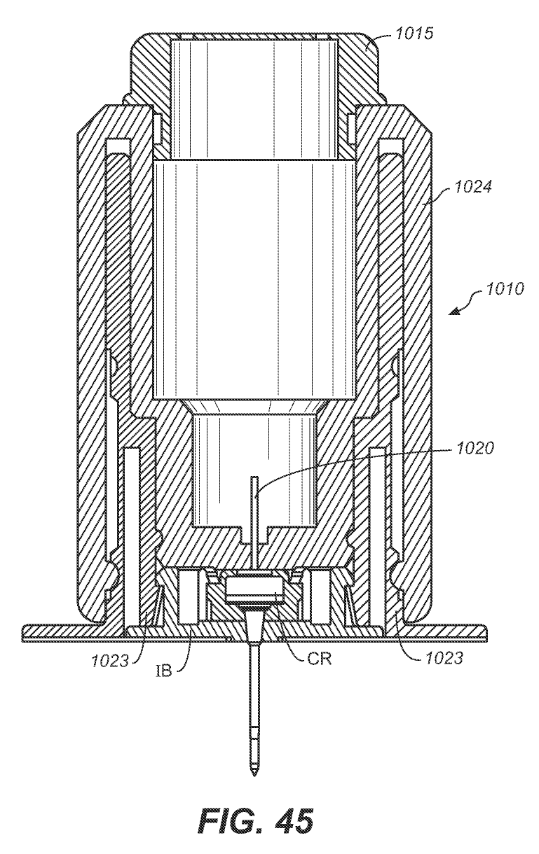

Cannula cartridge CR can be releasably retained using, for example, friction between cannula cartridge CR and cannula insertion needle 1020. Infuser base IB is retained in housing distal end by barbs 1023 of housing 1022 (see FIGS. 41 and 45). Barbs 1023 deflect when plunger 1024 is at a fully advanced position, thus releasing infuser base IB from retention.

Plunger 1024 is moveable from a retracted position, wherein a cannula cartridge retained on plunger distal 1030 end is remote from an infuser base releasably retained on housing distal end 1025 (see, for example, FIG. 41), to an advanced position (see, for example, FIG. 45) wherein the cannula cartridge CR is operatively engaged with an infuser base IB releasably retained on housing distal end 1025 and a cannula of a cannula cartridge has been subcutaneously inserted into an infusion site by cannula insertion needle 1020. In the embodiment of FIGS. 39-45, plunger movement from is accomplished in a manual manner by a user applying pressure to cap 1015.

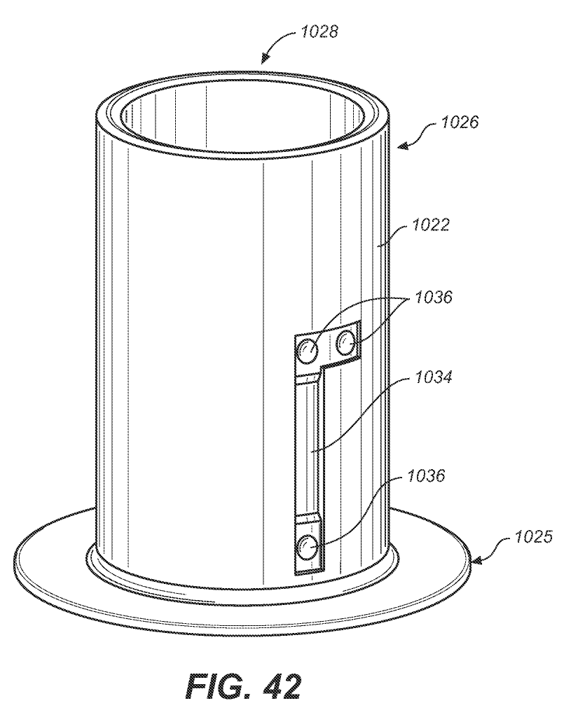

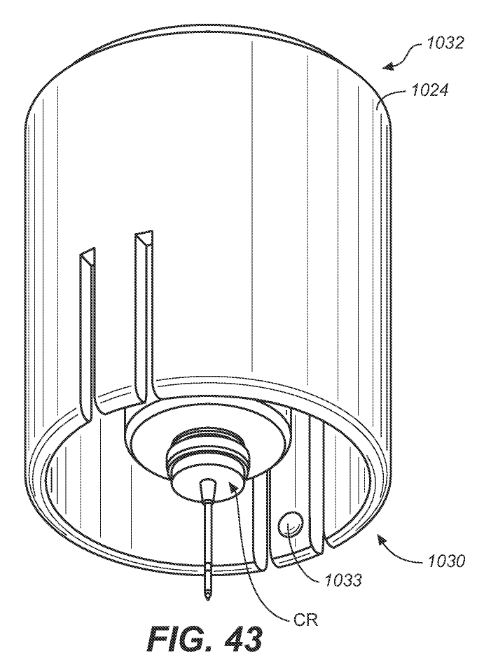

Plunger 1024 includes protrusions 1033 (one of which is visible in FIG. 43). Housing 1022 includes guide tracks 1034 with indentations 1036 (note that one guide track 1034 is visible in FIG. 42). During use of cannula insertion device 1000, protrusions 1033 and guide tracks 1034 mechanically cooperate such that plunger 1024 inserts cannula insertion needle 1020 perpendicularly into the infusion site without significant rotary motion. In addition, the most proximal portion of guide tracks 1034 provides a “locked” position wherein rotary but not perpendicular motion of the plunger is enabled. Moreover, the mechanical interaction of protrusions 1033 and indentations 1036 provide a user with tactile feedback during operation of cannula insertion device 1000. Although, for the purpose of illustration only, protrusions and indentations are depicted as hemispherical and circular in shape, any suitably shaped protrusions and indentations can be employed so long as they can cooperate with each other.

Housing distal end 1025 of housing 1022 is sized to apply pressure to an adhesive pad of infuser base IB and thus optimize adhesive attachment of infuser base IB to an infusion site when housing proximal end 1025 is urged against the infusion site prior to the moving of plunger 1024 to the advanced position.

Plunger 1024 and housing 1022 are configured such that plunger 1024 can be moved to the advanced position with a force below 0.5 lbf (i.e., 2.2238 Newtons). Therefore, a cannula can be easily inserted with only finger pressure. In addition, the speed of insertion is controlled by the user, insertion may be stopped at any time if the user feels discomfort. In addition, during use of cannula insertion device 1000, the infuser base is attached to the infusion site before insertion of the cannula, thus ensuring that the cannula is inserted perpendicular to the surface of the skin.

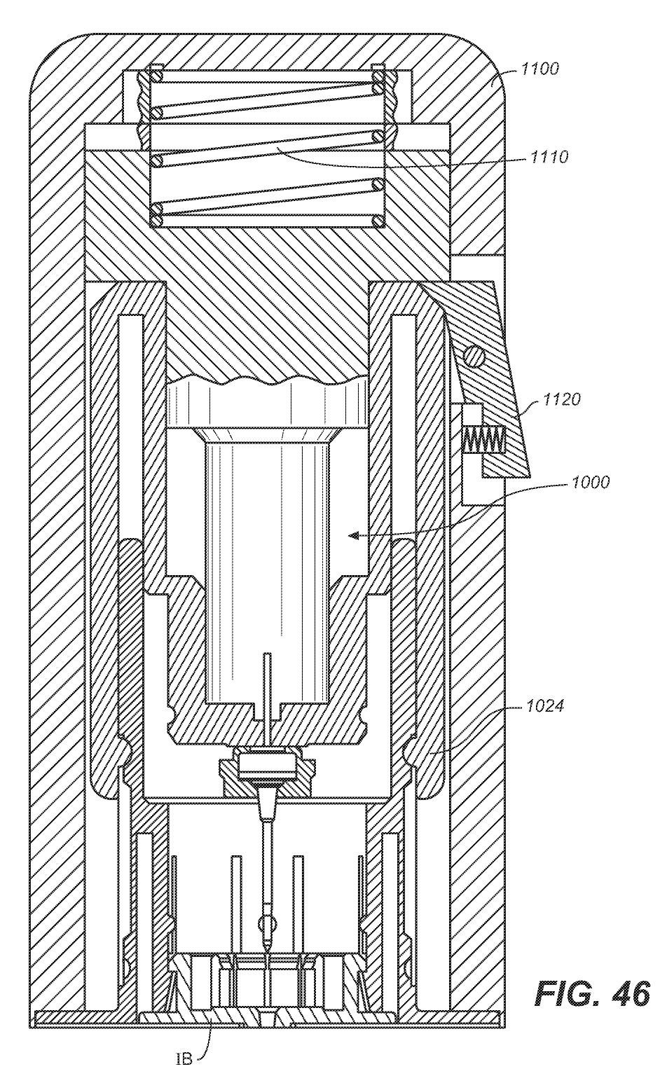

As an alternative to the manual operation of cannula insertion device 1000, cannula insertion device 1000 can be operated with an automatic insertion module. FIG. 46 is a simplified cross-sectional view of an automatic insertion module 1100 in use with cannula insertion device 1000 of FIGS. 39 through 45 absent cap 1015.

Automatic insertion module 1100 includes a drive spring 1110 and a latching mechanism 1120. Once armed, activation of automatic insertion module 1100 is accomplished by a user pressing latching mechanism 1120. Drive spring 1110 then advances plunger 1024 from a retracted position to an advanced position as previously described with respect to cannula insertion device 1000. Automatic insertion module 1100 can be armed prior to activation by, for example, a manual compression of drive spring 1110 and positioning of latching mechanism 1120 into the configuration depicted in FIG. 46.

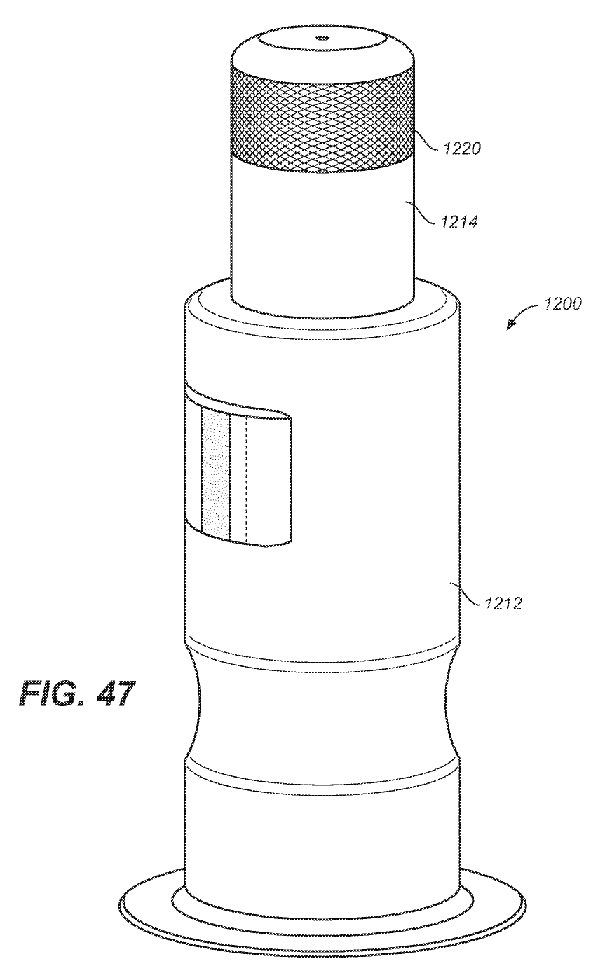

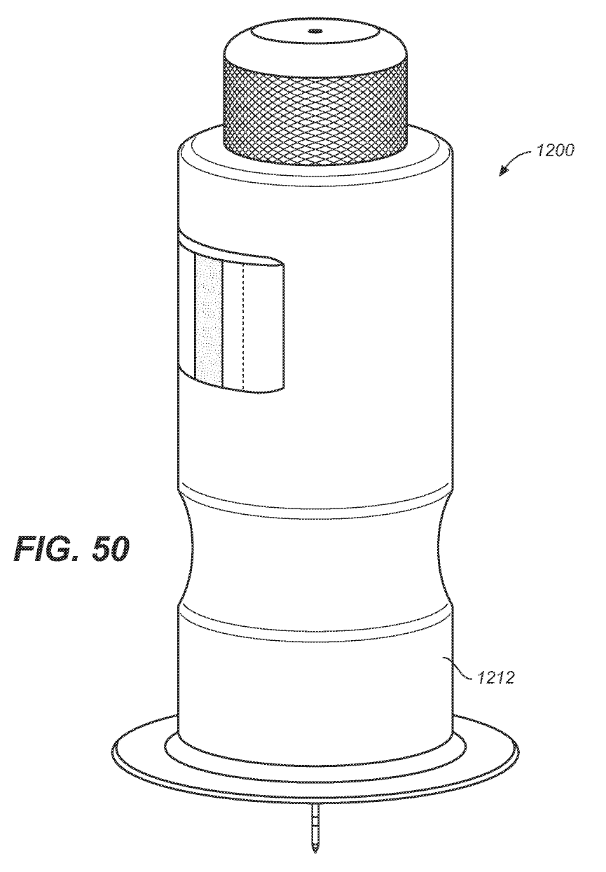

FIGS. 47 through 51B are various depictions of a cannula insertion device 1200 according to another exemplary embodiment of the present invention. Cannula insertion device 1200 includes an insertion module 1210 and a disposable cannula insertion needle cartridge 1220. Disposable cannula insertion needle cartridge 1220 can be removably locked into insertion module 1210 by any suitable means including, for example, a quarter-turn locking mechanism. Typically, disposable cannula insertion needle cartridge 1220 will have been provided with a sterile protective cover that is removed during use of cannula insertion device 1200. For example, such a sterile protective cover can be removed at an appropriate time after the cannula insertion needle cartridge has been locked into the insertion module.

Insertion module 1210 includes a housing 1212, a plunger 1214, a drive spring 1216 and a retainer cap 1218. Housing 1212 has a housing distal end 1224, a housing proximal end 1226 and a housing opening (not labeled in the FIGs.) extending from housing distal end 1224 to the housing proximal end 1226.

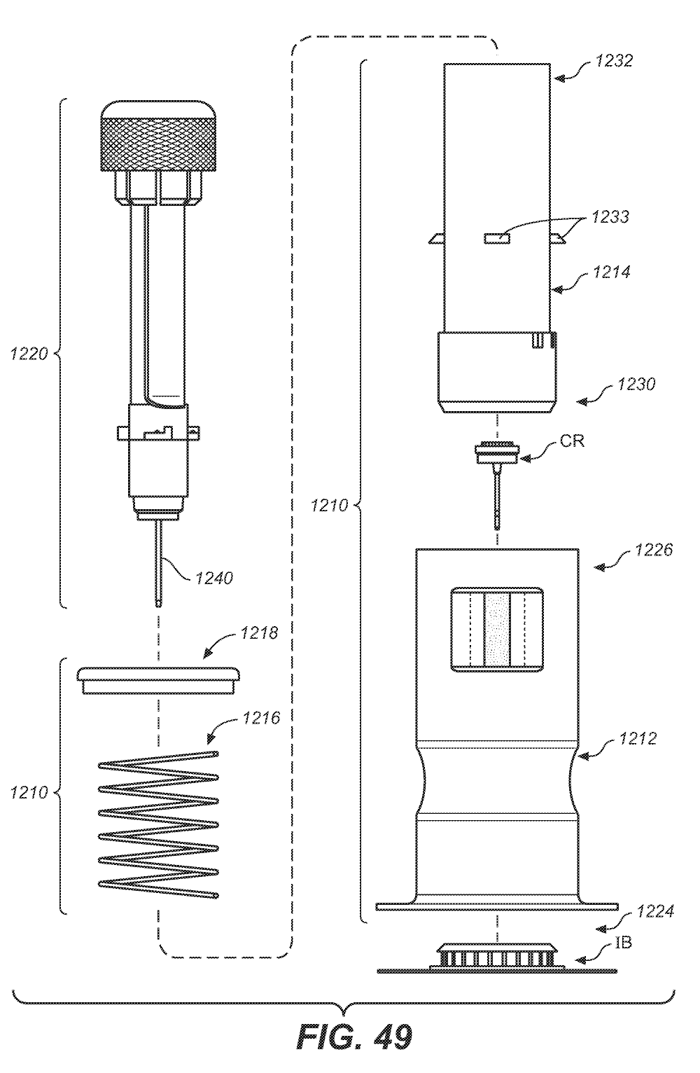

Plunger 1214 is disposed at least partially within the housing opening. Moreover, plunger 1214 includes a plunger distal end 1230, a plunger proximal end 1232 and barbs 1233. Cannula insertion needle 1240 is attached to a distal end of disposable cannula insertion needle cartridge 1220 (see FIG. 49).

The distal end of the plunger is configured to releasably retain a cannula cartridge CR of the infusion set in operative alignment with cannula insertion needle 1240. The housing distal end is configured to releasably retain an infuser base IB of the infusion set in operative alignment with the cannula cartridge CR.

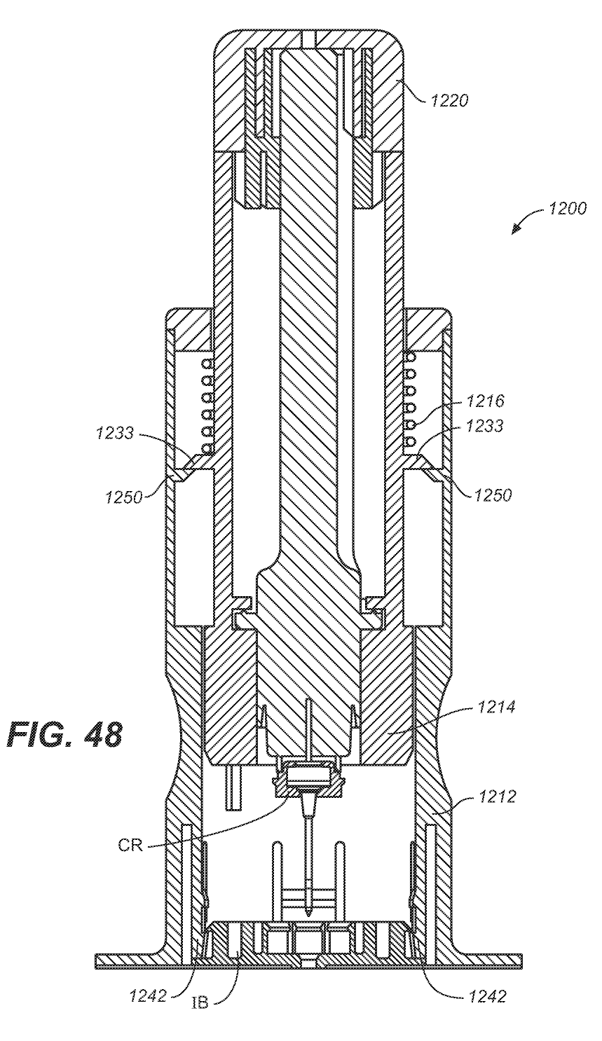

Cannula cartridge CR can be releasably retained using, for example, frictional engagement between cannula cartridge CR and cannula insertion needle 1240. Infuser base IB is retained in housing distal end by barbs 1242 of housing 1212 (see FIG. 48). Barbs 1242 deflect when plunger 1214 is at a fully advanced position, thus releasing infuser base IB from retention.

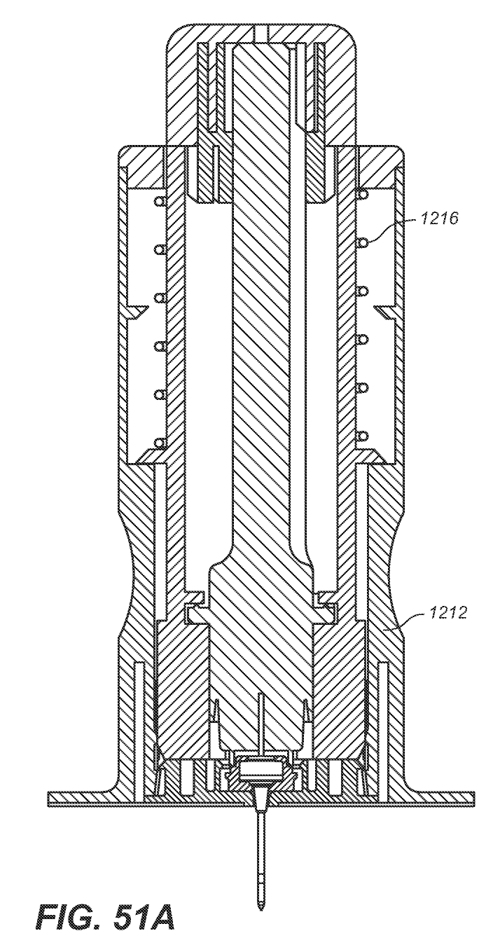

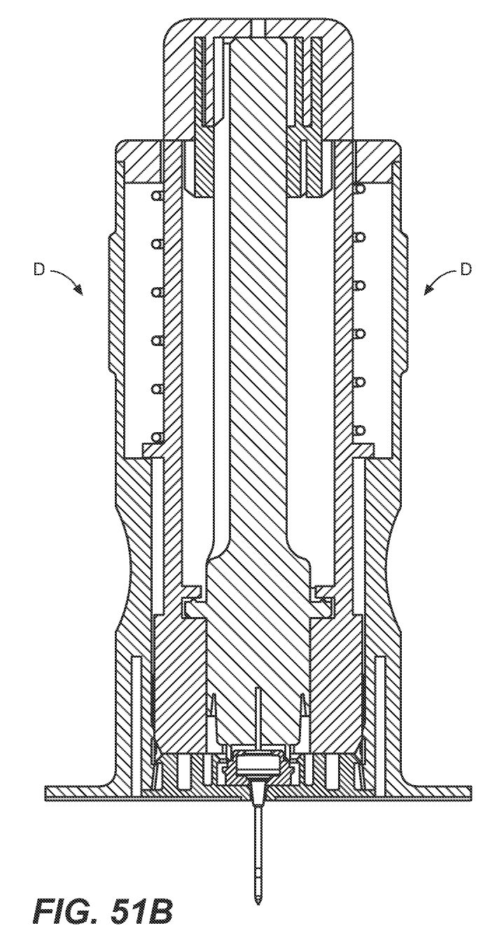

Plunger 1214 is moveable (under a force from drive spring 1216) from a retracted position, wherein a cannula cartridge retained on the plunger distal end is remote from an infuser base releasably retained on the housing distal end (see, for example, FIG. 48), to an advanced position (see, for example, FIGS. 51A and 51B) wherein the cannula cartridge CR is operatively engaged with an infuser base IB releasably retained on the housing distal end and a cannula of a cannula cartridge has been subcutaneously inserted into an infusion site by cannula insertion needle 1240.

Plunger 1024 is held in a retracted position by barbs 1233 engaging with barbs 1250 of housing 1212 (see FIG. 48). However, upon user application of pressure (e.g., a pressure in the range of approximately 0.25 to 4.0 lbf) to housing 1212 at the locations marked by arrows D in FIG. 51B, housing 1212 flexes and barbs 1233 and 1250 disengage. Upon such disengagement, drive spring 1216 moves plunger 1214 to the advanced position shown in FIGS. 51A and 51B.

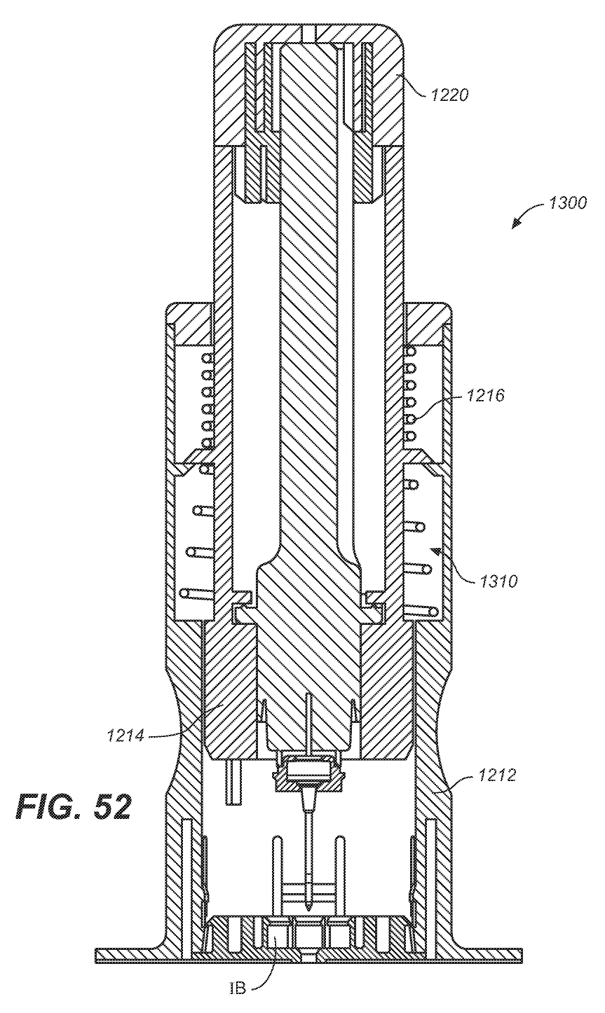

Drive spring 1216 is a unidirectional spring in that it serves only to move plunger 1214 to an advanced position. However, cannula insertion device 1200 can alternatively include a retraction spring. For example, FIG. 52 depicts a cannula insertion device 1300 that is essentially identical to cannula insertion device 1200 with the exception that retraction spring 1310 has been added to retract cannula insertion needle 1240 from the infusion site following insertion of a cannula.

Since disposable cannula insertion needle cartridge 1220 is removable, it can be safely disposed of while insertion module 1210 can be reused to effect cost savings. Moreover, since a simple flexing of housing 1212 is employed to activate drive spring 1216, no costly firing mechanisms are employed in cannula insertion devices 1200 and 1300.

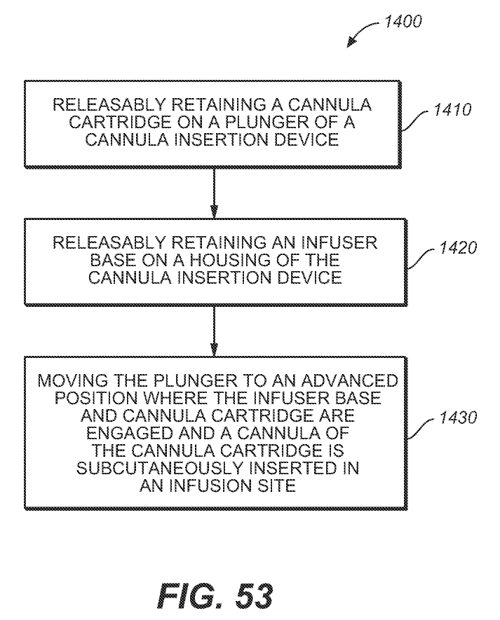

FIG. 53 is a flow diagram depicting stages in method 1400 for inserting a cannula of an infusion set into an insertion site according to an exemplary embodiment of the present invention. Method 1400 includes releasably retaining a cannula cartridge of an infusion set on a distal end of a plunger of a cannula insertion device, as set forth in step 1410.

At step 1420, an infuser base is releasably retained on a distal end of a housing of the cannula insertion device. Subsequently in step 1430, the plunger is moved from a retracted position, wherein the cannula cartridge retained on the plunger distal end is remote from the infuser base releasably retained on the housing distal end, to an advanced position wherein the cannula cartridge is engaged with the infuser base releasably retained on the housing distal end and a cannula of the cannula cartridge has been subcutaneously inserted into an infusion site by a cannula insertion needle attached to the plunger.

Once apprised of the present disclosure, one skilled in the art will recognize that method 1400 can be practiced using infusion sets and cannula insertion devices according to embodiments of the present invention. Therefore, functional characteristics, components, and benefits described with respect to infusion sets, infusion kits and cannula insertion devices according to the present invention can be incorporated into method 1400.

An infusion set kit according to embodiments of the present invention includes an infusion set and a cannula insertion device. The infusion set has an infuser base configured for adhesive attachment to an infusion site on a user's skin. The infusion set also has a cannula cartridge configured for operative engagement with the infuser base and a hub configured for user-controlled 360 degree (360°) rotary engagement with the infuser base.

Once apprised of the present disclosure, one skilled in the art will recognize infusion set kits according to embodiments of the present invention include the infusion sets and cannula insertion devices that have been described herein according to embodiments of the present invention. Therefore, functional characteristics, components, and benefits described with respect to those infusion sets and cannula insertion devices can be incorporated into the infusion sets of the present invention.

1. A method for inserting a cannula of an infusion set into an infusion site comprising:
releasably retaining a cannula cartridge of an infusion set on a distal end of a plunger of a cannula insertion device;
releasably retaining an infuser base on a distal end of a housing of the cannula insertion device;
moving the plunger from a retracted position, wherein the cannula cartridge retained on the plunger distal end is remote from an infuser base releasably retained on the housing distal end, to an advanced position wherein the cannula cartridge is engaged with the infuser base releasably retained on the housing distal end and a cannula of the cannula cartridge has been subcutaneously inserted into an infusion site by a cannula insertion needle attached to the plunger.

2. The method of claim 1 wherein the moving step employs a drive spring of the cannula insertion device in moving the plunger.

3. The method of claim 1 wherein a user manually moves the plunger.

4. The method of claim 1 wherein the moving step is activated by flexing of a housing of the cannula insertion device and the moving step employs a drive spring of the cannula insertion device to in moving the plunger.

5. The method of claim 1 further including the step of retracting the cannula insertion needle following the step of moving the plunger.

6. The method of claim 5 wherein the retracting step employs a retraction spring of the cannula insertion device.

7. The method of claim 1 wherein the moving step moves the plunger to an advanced position wherein the cannula has been perpendicularly subcutaneously inserted into the infusion site.
